(19) United States
(12) Patent Application Publication
(10) Pub. No.: US 2020/0171015 A1
(43) Pub. Date: Jun. 4, 2020

(54) METHODS OF TREATMENT FOR CYSTIC FIBROSIS

(71) Applicant: Vertex Pharmaceuticals Incorporated, Boston, MA (US)

(72) Inventors: Eric L. Haseltine, Melrose, MA (US); Samuel Moskowitz, Waban, MA (US); Sarah Robertson, Somerville, MA (US); David Waltz, Waban, MA (US)

(21) Appl. No.: 16/631,989

(22) PCT Filed: Jul. 17, 2018
(86) PCT No.: PCT/US2018/042415

§ 371 Date: Jan. 17, 2020

Related U.S. Application Data
(60) Provisional application No. 62/533,388, filed on Jul. 17, 2017.
(60) Provisional application No. 62/623,734, filed on Jan. 30, 2018.
(60) Provisional application No. 62/633,167, filed on Feb. 21, 2018.

(51) Int. Cl. A61K 31/4439; A61P 11/00; A61K 31/404; A61K 31/47

(52) U.S. Cl. CPC A61K 31/4439; A61K 31/47; A61K 31/404; A61P 11/00

(57) ABSTRACT

Methods of treating cystic fibrosis comprising administering at least Compound (I) of the formula. Pharmaceutical compositions containing a pharmaceutically acceptable salt of at least Compound I and methods of treating cystic fibrosis comprising administering a pharmaceutically acceptable salt of at least Compound (I).

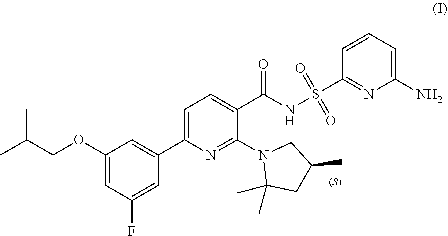

The instant application claims priority to U.S. Provisional Application No. 62/533,388, filed Jul. 17, 2017; U.S. Provisional Application No. 62/623,734, filed Jan. 30, 2018; and U.S. Provisional Application No. 62/633,167, filed Feb. 21, 2018, the entire contents of each of which are expressly incorporated herein by reference in their respective entireties.

Disclosed herein is a modulator of Cystic Fibrosis Transmembrane Conductance Regulator (CFTR), pharmaceutical compositions containing the modulator, methods of treatment of cystic fibrosis, and a process for making the modulator.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to enhanced mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 322 of these identified mutations, with sufficient evidence to define 281 mutations as disease causing. The most prevalent disease-causing mutation is a deletion of phenylalanine at position 508 of the CFTR amino acid sequence, and is commonly referred to as the F508del mutation. This mutation occurs in approximately 70% of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of approximately 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl− channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

Accordingly, there is a need for novel treatments of CFTR mediated diseases.

Disclosed herein is Compound I and pharmaceutically acceptable salts thereof. Compound I can be depicted as having the following structure:

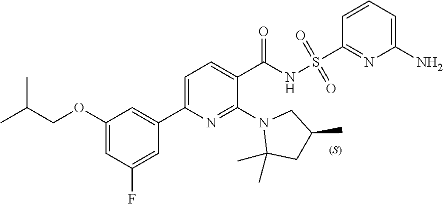

A chemical name for Compound I is N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide. PCT Publication No. WO 2016/057572, incorporated herein by reference, discloses Compound I, a method of making Compound I, and that Compound I is a CFTR modulator with an EC₃₀ of <3 μM.

Disclosed herein are pharmaceutical compositions wherein the properties of one therapeutic agent are improved by the presence of two therapeutic agents, kits, and methods of treatment thereof. In one embodiment, the disclosure features pharmaceutical compositions comprising N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I), (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), and N-[2,4-bis(1,1-dimethylethyl)-5-hydroxyphenyl]-1,4-dihydro-4-oxoquinoline-3-carboxamide (Compound III), wherein the composition has improved properties.

Also disclosed herein is a solid dispersion of N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I) in a polymer. In one embodiment, the solid dispersion is prepared by spray drying, and is referred to a spray-dried dispersion (SDD). In one embodiment, the spray dried dispersion has a Tg of from 80° C. to 180° C. In one embodiment, Compound I in the spray dried dispersion is substantially amorphous.

### BRIEF DESCRIPTION OF THE DRAWINGS

FIG. 1 is a representative list of CFTR genetic mutations.

As stated above, disclosed herein is Compound I, which can be depicted as having the following structure:

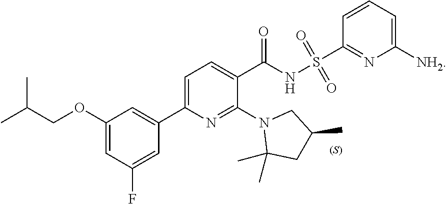

A chemical name for Compound I is N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide. Compound I may be in the form of a pharmaceutically acceptable salt thereof.

In some embodiments, Compound I (and/or at least one pharmaceutically acceptable salt thereof) can be administered in combination with at least one additional active pharmaceutical ingredient. In some embodiments, the at least one additional active pharmaceutical ingredient is chosen from:

(a) Compound II:

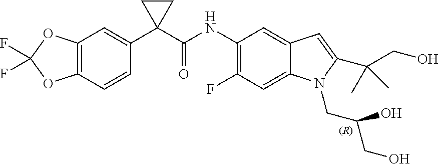

which has the following chemical name: (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, and pharmaceutically acceptable salts thereof; and

(b) Compound III:

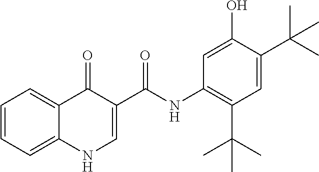 [SMILES: CC(C)(C)c1cc(C(C)(C)C)c(NC(=O)c2c[nH]c3ccccc3c2=O)cc1O]

which has the following chemical name: N-(5-hydroxy-2,4-di-tert-butyl-phenyl)-4-oxo-1H-quinoline-3-carboxamide, and pharmaceutically acceptable salts thereof, or

Compound III′:

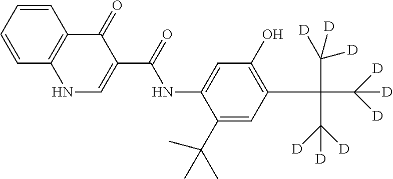 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

and pharmaceutically acceptable salts thereof.

### Definitions

As used herein, “CFTR” means cystic fibrosis transmembrane conductance regulator.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. A genetic defect or mutation, or a change in the nucleotides in a gene in general results in a mutation in the CFTR protein translated from that gene, or a frame shift(s).

The term “F508del” refers to a mutant CFTR protein which is lacking the amino acid phenylalanine at position 508.

As used herein, a patient who is “homozygous” for a particular gene mutation has the same mutation on each allele.

As used herein, a patient who is “heterozygous” for a particular gene mutation has this mutation on one allele, and a different mutation on the other allele.

As used herein, the term “modulator” refers to a compound that increases the activity of a biological compound such as a protein. For example, a CFTR modulator is a compound that increases the activity of CFTR. The increase in activity resulting from a CFTR modulator includes but is not limited to compounds that correct, potentiate, stabilize and/or amplify CFTR.

As used herein, the term “CFTR corrector” refers to a compound that facilitates the processing and trafficking of CFTR to increase the amount of CFTR at the cell surface. Compounds I and II disclosed herein are CFTR correctors.

As used herein, the term “CFTR potentiator” refers to a compound that increases the channel activity of CFTR protein located at the cell surface, resulting in enhanced ion transport. Compound III disclosed herein is a CFTR potentiator.

As used herein, the term “active pharmaceutical ingredient” or “therapeutic agent” (“API”) refers to a biologically active compound.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. Pharmaceutically acceptable salts are well known in the art. For example, S. M. Berge, et al. describe pharmaceutically acceptable salts in detail in J. Pharmaceutical Sciences, 1977, 66, 1-19.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment, and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of CF or its symptoms or lessening the severity of CF or its symptoms in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The term “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

Each of Compounds I, II, and III, and their pharmaceutically acceptable salts thereof independently can be administered once daily, twice daily, or three times daily. In some embodiments, at least one compound chosen from Compound I and pharmaceutically acceptable salts thereofthereof is administered once daily. In some embodiments, at least one compound chosen from Compound I and pharmaceutically acceptable salts thereofthereof are administered twice daily. In some embodiments, at least one compound chosen from Compound II and pharmaceutically acceptable salts thereof are administered once daily. In some embodiments, at least one compound chosen from Compound II and pharmaceutically acceptable salts thereof are administered twice daily. In some embodiments, at least one compound chosen from Compound III and pharmaceutically acceptable salts thereof is administered once daily. In some embodiments, at least one compound chosen from Compound III and pharmaceutically acceptable salts thereof are administered twice daily.

The term “daily” means per day. For example, 100 mg of Compound I is administered daily means total of 100 mg of Compound I per day is administered, which can be administered, for example, once daily, twice daily, or three times daily. For example, 100 mg of Compound I is administered once daily (qd) means 100 mg of Compound I per dosing is administered once per day. For example, 50 mg of Compound I is administered twice daily (bid) means 50 mg of Compound I per dosing is administered twice per day. In some embodiments, at least one compound chosen from Compound I and pharmaceutically acceptable salts thereof is administered once daily. In some embodiments, at least one compound chosen from Compound I and pharmaceutically acceptable salts thereof is administered twice daily. In some embodiments, Compound II or its pharmaceutically acceptable salts thereof are administered once daily. In some embodiments, Compound II or its pharmaceutically acceptable salts thereof are administered twice daily. In some embodiments, Compound III or its pharmaceutically acceptable salts thereof are administered once daily. In some embodiments, Compound III or its pharmaceutically acceptable salts thereof are administered twice daily. In some embodiments, Compound III-d or its pharmaceutically acceptable salts thereof are administered once daily. In some embodiments, Compound III-d or its pharmaceutically acceptable salts thereof are administered twice daily. In some embodiments, Compound IV or its pharmaceutically acceptable salts thereof are administered once daily. In some embodiments, Compound IV or its pharmaceutically acceptable salts thereof are administered twice daily.

In some embodiments, at least one compound chosen from Compound I and pharmaceutically acceptable salts thereof is administered in an amount from 50 mg to 1000 mg, 100 mg to 800 mg, 100 mg to 700 mg, 100 mg to 600 mg, 200 mg to 600 mg, 300 mg to 600 mg, 400 mg to 600 mg, 500 mg to 700 mg, or 500 mg to 600 mg, daily. In some embodiments, at least one compound chosen from Compound I and pharmaceutically acceptable salts thereof is administered in an amount of 100 mg, 200 mg, 300 mg, 400 mg, 500 mg, 600 mg, 700 mg, or 800 mg, daily. In some embodiments, at least one compound chosen from Compound I and pharmaceutically acceptable salts thereof are administered in an amount of 100 mg, 200 mg, 300 mg, 400 mg, 500 mg, 600 mg, 700 mg, 800 mg, 900 mg, or 1000 mg, once daily. In some embodiments, at least one compound chosen from Compound I and pharmaceutically acceptable salts thereof is administered in an amount of 50 mg, 100 mg, 150 mg, 200 mg, 250 mg, 300 mg, 350 mg, 400 mg, 450 mg, or 500 mg, twice daily.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form. For example, “100 mg of at least one compound chosen from Compound I and pharmaceutically acceptable salts thereof” includes 100 mg of Compound I and a concentration of pharmaceutically acceptable salt of Compound I equivalent to 100 mg of Compound I.

Compounds I, II, and III, and their pharmaceutically acceptable salts thereof can be comprised in a single pharmaceutical composition or separate pharmaceutical compositions. Such pharmaceutical compositions can be administered once daily or multiple times daily, such as twice daily.

In some embodiments, at least one compound chosen from Compound I and pharmaceutically acceptable salts thereof is comprised in a first pharmaceutical composition; at least one compound chosen from Compound II and pharmaceutically acceptable salts thereof is comprised in a second pharmaceutical composition; and at least one compound chosen from Compound III and pharmaceutically acceptable salts thereof is comprised in a third pharmaceutical composition.

In some embodiments, at least one compound chosen from Compound I and pharmaceutically acceptable salts thereof is comprised in a first pharmaceutical composition; and at least one compound chosen from Compound II and pharmaceutically acceptable salts thereof and at least one compound chosen from Compound III and pharmaceutically acceptable salts thereof are comprised in a second pharmaceutical composition. In some embodiments, the second pharmaceutical composition comprises a half of the daily dose of said at least one compound chosen from Compound III and pharmaceutically acceptable salts thereof, and the other half of the daily dose of said at least one compound chosen from Compound III and pharmaceutically acceptable salts thereof is administered in a third pharmaceutical composition.

In some embodiments, at least one compound chosen from Compound I and pharmaceutically acceptable salts thereof is comprised in a first pharmaceutical composition; at least one compound chosen from Compound II and pharmaceutically acceptable salts thereof; and at least one compound chosen from Compound III and pharmaceutically acceptable salts thereof are comprised in a first pharmaceutical composition. In some embodiments, the first pharmaceutical composition is administered to the patient twice daily.

In some embodiments, the disclosure features a pharmaceutical composition comprising at least one compound chosen from Compound I and pharmaceutically acceptable salts thereof, and at least one pharmaceutically acceptable carrier.

In some embodiments, the disclosure features a pharmaceutical composition comprising at least one compound chosen from Compound I and pharmaceutically acceptable salts thereof, at least one compound chosen from Compound II and pharmaceutically acceptable salts thereof, and at least one pharmaceutically acceptable carrier.

In some embodiments, the disclosure features a pharmaceutical composition comprising at least one compound chosen from Compound I and pharmaceutically acceptable salts thereof, at least one compound chosen from Compound III and pharmaceutically acceptable salts thereof, and at least one pharmaceutically acceptable carrier.

In some embodiments, the disclosure features a pharmaceutical composition comprising at least one compound chosen from Compound I and pharmaceutically acceptable salts thereof, at least one compound chosen from Compound II and pharmaceutically acceptable salts thereof, at least one compound chosen from Compound III and pharmaceutically acceptable salts thereof, and at least one pharmaceutically acceptable carrier.

In some embodiments, pharmaceutical compositions disclosed herein comprise at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises Compound I and at least two additional active pharmaceutical ingredients, one of which is a CFTR corrector and one of which is a CFTR potentiator.

In some embodiments, at least one additional active pharmaceutical ingredient is selected from mucolytic agents, bronchodilators, antibiotics, anti-infective agents, and anti-inflammatory agents.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

It will also be appreciated that a pharmaceutical composition of this disclosure, including a pharmaceutical composition comprising combinations described previously, can be employed in combination therapies; that is, the compositions can be administered concurrently with, prior to, or subsequent to, at least one additional active pharmaceutical ingredient or medical procedures.

In some embodiments, a pharmaceutical composition disclosed herein comprises at least one compound chosen from Compound I and pharmaceutically acceptable salts thereof, and at least one pharmaceutically acceptable carrier. In some embodiments, the pharmaceutically acceptable carrier is a polymer. In some embodiments, the pharmaceutically acceptable carrier is HPMCAS. In some embodiments, the pharmaceutically acceptable carrier is HPMCAS-H. In some embodiments, the pharmaceutical composition comprises a solid dispersion of compound I in HPMCAS-H.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutically acceptable compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutically acceptable composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

It will also be appreciated that a pharmaceutical composition of this disclosure, including a pharmaceutical composition comprising any of the combinations described previously, can be employed in combination therapies; that is, the compositions can be administered concurrently with, prior to, or subsequent to, at least one active pharmaceutical ingredients or medical procedures.

In some embodiments, the methods disclosed herein employ administering to a patient in need thereof at least one compound chosen from Compound I and pharmaceutically acceptable salts thereof; and at least one selected from Compound II, Compound III, and pharmaceutically acceptable salts thereof.

Any suitable pharmaceutical compositions known in the art can be used for Compound I, Compound II, Compound III, and pharmaceutically acceptable salts thereof. Some exemplary pharmaceutical compositions for Compound I and its pharmaceutically acceptable salts are described in the Examples. Some exemplary pharmaceutical compositions for Compound II and its pharmaceutically acceptable salts can be found in WO 2011/119984 and WO 2014/015841, both of which are incorporated herein by reference. Some exemplary pharmaceutical compositions for Compound III and its pharmaceutically acceptable salts can be found in WO 2007/134279, WO 2010/019239, WO 2011/019413, WO 2012/027731, and WO 2013/130669, all of which are incorporated herein by reference.

In some embodiments, a pharmaceutical composition comprising at least one compound chosen from Compound I and pharmaceutically acceptable salts thereof is administered with a pharmaceutical composition comprising Compound II and Compound III. Pharmaceutical compositions comprising Compound II and Compound III are disclosed in PCT Publication No. WO 2015/160787, incorporated herein by reference. An exemplary embodiment is shown in the following Table:

TABLE 2
Exemplary Tablet Comprising 100 mg Compound
II and 150 mg Compound III.

| | | Amount per |
|---|---|---|
| | Ingredient | tablet (mg) |
| Intra-granular | Compound II SDD (spray | 125 |
| | dried dispersion) | |
| | (80 wt % Compound II; 20 | |
| | wt % HPMC) | |
| | Compound III SDD | 187.5 |
| | (80 wt % Compound III; | |
| | 19.5 wt % HPMCAS-HG; | |
| | 0.5 wt % sodium lauryl | |
| | sulfate) | |
| | Microcrystalline cellulose | 131.4 |
| | Croscarmellose Sodium | 29.6 |
| | Total | 473.5 |
| Extra-granular | Microcrystalline cellulose | 112.5 |
| | Magnesium Stearate | 5.9 |
| | Total | 118.4 |
| Total uncoated Tablet | | 591.9 |
| Film coat | Opadry | 17.7 |
| Total coated Tablet | | 609.6 |

In some embodiments, a pharmaceutical composition comprising Compound I is administered with a pharmaceutical composition comprising Compound III. Pharmaceutical compositions comprising Compound III are disclosed in PCT Publication No. WO 2010/019239, incorporated herein by reference. An exemplary embodiment is shown in the following Table:

TABLE 3
Ingredients for Exemplary Tablet of Compound III.

| | Percent Dose | Dose | Batch |
|---|---|---|---|
| Tablet Formulation | % Wt./Wt. | (mg) | (g) |
| Compound III SDD | 34.09% | 187.5 | 23.86 |
| (80 wt % Compound III; 19.5 wt % | | | |
| HPMCAS-HG; 0.5 wt % sodium | | | |
| lauryl sulfate) | | | |
| Microcrystalline cellulose | 30.51% | 167.8 | 21.36 |
| Lactose | 30.40% | 167.2 | 21.28 |
| Sodium croscarmellose | 3.000% | 16.50 | 2.100 |
| SLS | 0.500% | 2.750 | 0.3500 |
| Colloidal silicon dioxide | 0.500% | 2.750 | 0.3500 |
| Magnesium stearate | 1.000% | 5.500 | 0.7000 |
| Total | 100% | 550 | 70 |

Additional pharmaceutical compositions comprising Compound III are disclosed in PCT Publication No. WO 2013/130669, incorporated herein by reference. Exemplary mini-tablets (˜2 mm diameter, ˜2 mm thickness, each mini-tablet weighing about 6.9 mg) was formulated to have approximately 50 mg of Compound III per 26 mini-tablets and approximately 75 mg of Compound III per 39 mini-tablets using the amounts of ingredients recited in Table 4, below.

TABLE 4
Ingredients for mini-tablets for 50 mg and 75 mg potency

| | Percent | Dose (mg) | Dose (mg) | |
|---|---|---|---|---|
| | Dose | 50 mg | 75 mg | Batch |
| Tablet Formulation | % Wt./Wt. | potency | potency | (g) |
| Compound III SDD | 35 | 62.5 | 93.8 | 1753.4 |
| (80 wt % Compound | | | | |
| III; 19.5 wt % | | | | |
| HPMCAS-HG; 0.5 | | | | |
| wt % sodium lauryl | | | | |
| sulfate) | | | | |
| Mannitol | 13.5 | 24.1 | 36.2 | 675.2 |
| Lactose | 41 | 73.2 | 109.8 | 2050.2 |
| Sucralose | 2.0 | 3.6 | 5.4 | 100.06 |
| Croscarmellose sodium | 6.0 | 10.7 | 16.1 | 300.1 |
| Colloidal silicon | 1.0 | 1.8 | 2.7 | 50.0 |
| dioxide | | | | |
| Magnesium stearate | 1.5 | 2.7 | 4.0 | 74.19 |
| Total | 100 | 178.6 | 268 | 5003.15 |

In some embodiments, the pharmaceutical compositions are a tablet. In some embodiments, the tablets are suitable for oral administration.

These combinations are useful for treating cystic fibrosis.

A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect).

In some embodiments, disclosed herein methods of treating, lessening the severity of, or symptomatically treating cystic fibrosis in a patient comprising administering an effective amount of a compound, pharmaceutically acceptable salt thereof, or a deuterated analog of any of the foregoing; or a pharmaceutical composition, of this disclosure to a patient, such as a human, wherein said patient has cystic fibrosis. In some embodiments, the patient has F508del/minimal function (MF) genotypes, F508del/F508del genotypes, F508del/gating genotypes, or F508del/residual function (RF) genotypes.

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance. Table C below includes a non-exclusive list of CFTR minimal function mutations, which are detectable by an FDA-cleared genotyping assay. In some embodiments, a mutation is considered a MF mutation if it meets at least 1 of the following 2 criteria:
(1) biological plausibility of no translated protein (genetic sequence predicts the complete absence of CFTR protein), or
(2) in vitro testing that supports lack of responsiveness to Compound II, Compound III or the combination of Compound II and Compound III, and evidence of clinical severity on a population basis (as reported in large patient registries).

In some embodiments, the minimal function mutations are those that result in little-to-no functioning CFTR protein and are not responsive in vitro to Compound II, Compound III, or the combination of Compound II and Compound III.

In some embodiments, the minimal function mutations are those that are not responsive in vitro to Compound II, Compound III, or the combination of Compound II and Compound III. In some embodiments, the minimal function mutations are mutations based on in vitro testing met the following criteria in in vitro experiments:
baseline chloride transport that was <10% of wildtype CFTR, and
an increase in chloride transport of <10% over baseline following the addition of TEZ, IVA, or TEZ/IVA in the assay.

In some embodiments, patients with at least one minimal function mutation exhibit evidence of clinical severity as defined as:
average sweat chloride >86 mmol/L, and
prevalence of pancreatic insufficiency (PI)>50%.

Patients with an F508del/minimal function genotype are defined as patients that are heterozygous F508del-CFTR with a second CFTR allele containing a a minimal function mutation. In some embodiments, patients with an F508del/minimal function genotype are patients that are heterozygous F508del-CFTR with a second CFTR allele containing a mutation that results in a CFTR protein with minimal CFTR function (little-to-no functioning CFTR protein) and that is responsive in vitro to Compound II, Compound III, or the combination of Compound II and Compound III.

In some embodiments, minimal function mutations can be using 3 major sources:
biological plausibility for the mutation to respond (i.e., mutation class)
evidence of clinical severity on a population basis (per CFTR2 patient registry; accessed on 15 Feb. 2016)
   average sweat chloride >86 mmol/L, and
   prevalence of pancreatic insufficiency (PI)>50%
in vitro testing
   mutations resulting in baseline chloride transport <10% of wild-type CFTR were considered minimal function
   mutations resulting in chloride transport <10% of wild-type CFTR following the addition of Compound II and/or Compound III were considered nonresponsive.

As used herein, a “residual function mutations” refer to are Class II through V mutations that have some residual chloride transport and result in a less severe clinical phenotype. Residual function mutations are mutation in the CFTR gene that result in reduced protein quantity or function at the cell surface which can produce partial CFTR activity.

Non-limiting examples of CFTR gene mutations known to result in a residual function phenotype include a CFTR residual function mutation selected from 2789+5G→A, 3849+1 OkbC→T, 3272-26A→G, 711+3A→G, E56K, P67L, R74W, DllOE, DL LOH, R117C, L206W, R347H, R352Q, A455E, D579G, E831X, S945L, S977F, F1052V, R1070W, F1074L, Dl 152H, D1270N, El93K, and Kl060T. For example, CFTR mutations that cause defective mRNA splicing, such as 2789+507 A, result in reduced protein synthesis, but deliver some functional CFTR to the surface of the cell to provide residual function. Other CFTR mutations that reduce conductance and/or gating, such as R1 17H, result in a normal quantity of CFTR channels at the surface of the cell, but the functional level is low, resulting in residual function. In some embodiments, the CFTR residual function mutation is selected from R117H, S1235R, I1027T, R668C, G576A, M470V, L997F, R75Q, R1070Q, R31C, D614G, G1069R, R1162L, E56K, A1067T, E193K, and K1060T. In some embodiments, the CFTR residual function mutation is selected from R117H, S1235R, I1027T, R668C, G576A, M470V, L997F, R75Q, R1070Q, R31C, D614G, G1069R, R1162L, E56K, and A1067T.

Residual CFTR function can be characterized at the cellular (in vitro) level using cell based assays, such as an FRT assay (Van Goar, F. et al. (2009) PNAS Vol. 106, No. 44, 18825-18830; and Van Goor, F. et al. (2011) PNAS Vol. 108, No. 46, 18843-18846), to measure the amount of chloride transport through the mutated CFTR channels. Residual function mutations result in a reduction but not complete elimination of CFTR dependent ion transport. In some embodiments, residual function mutations result in at least about 10% reduction of CFTR activity in an FRT assay. In some embodiments, the residual function mutations result in up to about 90% reduction in CFTR activity in an FRT assay.

Patients with an F508del/residual function genotype are defined as patients that are heterozygous F508del-CFTR with a second CFTR allele that contains a mutation that results in reduced protein quantity or function at the cell surface which can produce partial CFTR activity.

Patients with an F508del/gating mutation genotype are defined as patients that are heterozygous F508del-CFTR with a second CFTR allele that contains a mutation associated with a gating defect and clinically demonstrated to be responsive to Compound III. Examples of such mutations include: G178R, S549N, S549R, G551D, G551S, G1244E, S1251N, S1255P, and G1349D.

In some embodiments, the methods of treating, lessening the severity of, or symptomatically treating cystic fibrosis disclosed herein are each independently produces an increase in chloride transport above the baseline chloride transport of the patient.

In some embodiments, in the methods of treating, lessening the severity of, or symptomatically treating cystic fibrosis disclosed herein, the patient is heterozygous for F508del, and the other CFTR genetic mutation is any CF-causing mutation. In some embodiments, the patient is heterozygous for F508del, and the other CFTR genetic mutation is any CF-causing mutation, and is expected to be and/or is responsive to any of the compounds disclosed herein, such as Compound 1, Compound II, and/or Compound III genotypes based on in vitro and/or clinical data. In some embodiments, the patient is heterozygous for F508del, and the other CFTR genetic mutation is any CF-causing mutation, and is expected to be and/or is responsive to any combinations of (i) Compound 1, and (ii) Compound II, and/or Compound III and/or Compound IV genotypes based on in vitro and/or clinicCompound IVal data.

In some embodiments, in the methods of treating, lessening the severity of, or symptomatically treating cystic fibrosis disclosed herein, the patient possesses a CFTR genetic mutation selected from any of the mutations listed in Table A.

TABLE A
CF Mutations

078delT
1078delT
11234V
1154insTC
1161delC
1213delT
1248+1G→A
1249−1G→A
124del23bp
1259insA
1288insTA
1341+1G−>A
1342−2A−>C
1461ins4
1471delA
1497delGG
1507del
1525−1G→A
1525−2A→G
1548delG
1577delTA
1609del CA
1677delTA
1716G/A
1717−1G→A
1717−8G→A
1782delA
1811+1.6kbA−>G
1811+1G−>C
1811+1.6kbA→G
1811+1G→C
1812−1G−>A
1898+1G−>A
1812−1G→A
1824delA
182delT 1119delA
185+1G→T
1898+1G−>T
1898+1G→A
1898+1G→C
1898+3A−>G
1898+5G−>T
1924del7
1949del84
2043delG
2055del9→A
2105-2117del13insAGAAA
2118del14
2143delT
2183AA−>G+
2183AA→G
2183AA→G^{a}
2183delAA−>G#
2183delAA→G
2184delA
2184insA
2307insA
2347delG
2556insAT
2585delT
2594delGT
2622+1G−>A
2622+lG−>A
2659delC
2711delT
271delT
2721del11
2732insA
2789+2insA
2789+5G→A
2790−1G→C
2790−IG−>C
2869insG
2896insAG
2942insT
2957delT
296+1G→A
2991del32
3007delG
3028delA
3040G→C
306insA
306insA
1138insG
3120G→A
3121−1G→A
3121−2A→G
3121−977_3499+248del2515
3132delTG
3141del9
3171delC
3195del6
3199del6
3272−26A−>G
3500−2A→G
3600+2insT
365−366insT
3659delC
3667ins4
3737delA
3791delC
3821delT
3849+10kbC→T
3849+IOkbC−>T
3850−1G→A
3850−3T−>G
3850−lG−>A
3876delA
3878delG
3905InsT
3905insT
394delTT
4005+1G−>A
4005+2T−>C
4005+1G→A
4005+lG−>A
4010del4
4015delA
4016insT
4021dupT
4040delA
405+1G→A
405+3A→C
405+IG−>A
406−1G→A
406−IG−>A
4209TGTT−>A
4209TGTT→AA
4279insA
4326delTC
4374+1G→T
4374+IG−>T
4382delA
4428insGA
442delA
457TAT→G
541delC
574delA
5T
621+1G→T
621+3A−>G
663delT
663delT 1548delG
675del4
711+1G−>T
711+3A−>G
711+1G→T
711+3A→G
711+5G→A
712−1G−>T
7T
852del22
935delA
991del5
A1006E
A120T
A234D
A349V
A455E
A613T
A46D
A46Db
A559T
A559Tb
A561E
C276X
C524R
C524X
CFTRdel2,3
CFTRdele22-23
D110E
D110H
D1152H
D1270N
D192G
D443Y
D513G
D579G
D614G
D836Y
D924N
D979V
E1104X
E116K
E1371X
E193K
E193X
E403D
E474K
E56K
E585X
E588V
E60K
E822K
E822X
E831X
E92K
E92X
F1016S
F1052V
F1074L
F1099L
F191V
F311del
F311L
F508C
F508del
F575Y
G1061R
G1069R
G1244E
G1249R
G126D
G1349D
G149R
G178R
G194R
G194V
G27R
G27X
G314E
G330X
G458V
G463V
G480C
G542X
G550X
G551D
G551S
G576A
G622D
G628R
G628R(G−>A)
G970D
G673X
G85E
G91R
G970R
G970R
H1054D
H1085P
H1085R
H1375P
H139R
H199R
H199Y
H609R
H939R
I1005R
I1027T
I1234V
I1269N
I1366N
I148T
I175V
I3336K
I502T
I506S
I506T
I507del
I507del
I601F
I618T
I807M
I980K
IVS14b+5G−>A
K710X
K710X
K710X
L102R
L1065P
L1077P
L1077Pb
L1254X
L1324P
L1335P
L138ins
L1480P
L15P
L165S
L206W
L218X
L227R
L320V
L346P
L453S
L467P
L467Pb
L558S
L571S
L732X
L927P
L967S
L997F
M1101K
M1101R
M152V
M1T
M1V
M265R
M470V
M952I
M952T
N1303K
P205S
P574H
P5L
P67L
P750L
P99L
Q1100P
Q1291H
Q1291R
Q1313X
Q1382X
Q1411X
Q1412X
Q220X
Q237E
Q237H
Q452P
Q290X
Q359K/T360K
Q39X
Q414
Q414X
E585X
Q493X
Q525X
Q552X
Q685X
Q890X
Q890X
Q98R
Q98X
R1066C
R1066H
R1066M
R1070Q
R1070W
R1102X
R1158X
R1162L
R1162X
R117C
R117G
R117H
R117L
R117P
R1283M
R1283S
R170H
R258G
R31C
R31L
R334L
R334Q
R334W
R347H
R347L
R347P
R352Q
R352W
R516G
R553Q
R553X
R560K
R560S
R560T
R668C
R709X
R74W
R751L
R75Q
R75X
R764X
R792G
R792X
R851X
R933G
S1118F
S1159F
S1159P
S1196X
S1235R
S1251N
S1255P
S1255X
S13F
S341P
S434X
S466X
S489X
S492F
S4X
S549N
S549R
S549R(A−>C)
S549R(T−>G)
S589N
S737F
S912L
S912X
S945L
S977F
T1036N
T1053I
T1246I
T338I
T604I
V1153E
V1240G
V1293G
V201M
V232D
V456A
V456F
V520F
V562I
V754M
W1089X
W1098C
W1098R
W1098X
W1204X
W1282R
W1282X
W361R
W401X
W496X
W57G
W57R
W57X
W846X
Y1014C
Y1032C
Y1092X
Y109N
Y122X
Y161D
Y161S
Y563D
Y563N
Y569C
Y569D
Y569Db
Y849X
Y913C
Y913X

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, 621+3A->G, 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, I1005R, I1234V, I11269N, I1366N, I175V, I502T, I506S, I506T, I601F, I618T, I807M, I980K, L102R, L1324P, L1335P, L138ins, L1480P, L15P, L165S, L320V, L346P, L453S, L571S, L967S, M1101R, M152V, M1T, M1V, M265R, M952I, M952T, P574H, P5L, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T1053I, T1246I, T604I, V1153E, V1240G, V1293G, V201M, V232D, V456A, V456F, V562I, W1098C, W1098R, W1282R, W361R, W57G, W57R, Y1014C, Y1032C, Y109N, Y161D, Y161S, Y563D, Y563N, Y569C, and Y913C.

In some embodiments, the patient has at least one combination mutation chosen from: G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, and 621+3A->G.

In some embodiments, the patient has at least one combination mutation chosen from: 1949del84, 3141del9, 3195del6, 3199del6, 3905InsT, 4209TGTT->A, A1006E, A120T, A234D, A349V, A613T, C524R, D192G, D443Y, D513G, D836Y, D924N, D979V, E116K, E403D, E474K, E588V, E60K, E822K, F1016S, F1099L, F191V, F311del, F311L, F508C, F575Y, G1061R, G1249R, G126D, G149R, G194R, G194V, G27R, G314E, G458V, G463V, G480C, G622D, G628R, G628R(G->A), G91R, G970D, H1054D, H1085P, H1085R, H1375P, H139R, H199R, H609R, H939R, I1005R, I1234V, I11269N, I1366N, I175V, I502T, I506S, I506T, I601F, I618T, I807M, I980K, L102R, L1324P, L1335P, L138ins, L1480P, L15P, L165S, L320V, L346P, L453S, L571S, L967S, M1101R, M152V, M1T, M1V, M265R, M952I, M952T, P574H, P5L, P750L, P99L, Q1100P, Q1291H, Q1291R, Q237E, Q237H, Q452P, Q98R, R1066C, R1066H, R117G, R117L, R117P, R1283M, R1283S, R170H, R258G, R31L, R334L, R334Q, R347L, R352W, R516G, R553Q, R751L, R792G, R933G, S1118F, S1159F, S1159P, S13F, S549R(A->C), S549R(T->G), S589N, S737F, S912L, T1036N, T1053I, T1246I, T604I, V1153E, V1240G, V1293G, V201M, V232D, V456A, V456F, V562I, W1098C, W1098R, W1282R, W361R, W57G, W57R, Y1014C, Y1032C, Y109N, Y161D, Y161S, Y563D, Y563N, Y569C, and Y913C.

In some embodiments, in the methods of treating, lessening the severity of, or symptomatically treating cystic fibrosis disclosed herein, the patient possesses a CFTR genetic mutation G551D. In some embodiments, the patient is homozygous for the G551D genetic mutation. In some embodiments, the patient is heterozygous for the G551D genetic mutation. In some embodiments, the patient is heterozygous for the G551D genetic mutation, having the G551D mutation on one allele and any other CF-causing mutation on the other allele. In some embodiments, the patient is heterozygous for the G551D genetic mutation on one allele and the other CF-causing genetic mutation on the other allele is any one of F508del, G542X, N1303K, W1282X, R117H, R553X, 1717-1G->A, 621+1G->T, 2789+5G->A, 3849+10kbC->T, R1162X, G85E, 3120+1G->A, ΔI507, 1898+1G->A, 3659delC, R347P, R560T, R334W, A455E, 2184delA, or 711+1G->T. In some embodiments, the patient is heterozygous for the G551D genetic mutation, and the other CFTR genetic mutation is F508del. In some embodiments, the patient is heterozygous for the G551D genetic mutation, and the other CFTR genetic mutation is R117H.

In some embodiments, in the methods of treating, lessening the severity of, or symptomatically treating cystic fibrosis disclosed herein, the patient possesses a CFTR genetic mutation F508del. In some embodiments, the patient is homozygous for the F508del genetic mutation. In some embodiments, the patient is heterozygous for the F508del genetic mutation wherein the patient has the F508del genetic mutation on one allele and any CF-causing genetic mutation on the other allele. In some embodiments, the patient is heterozygous for F508del, and the other CFTR genetic mutation is any CF-causing mutation, including, but not limited to G551D, G542X, N1303K, W1282X, R117H, R553X, 1717-1G->A, 621+1G->T, 2789+5G->A, 3849+10kbC->T, R1162X, G85E, 3120+1G->A, ΔI507, 1898+1G->A, 3659delC, R347P, R560T, R334W, A455E, 2184delA, or 711+1G->T. In some embodiments, the patient is heterozygous for F508del, and the other CFTR genetic mutation is G551D. In some embodiments, the patient is heterozygous for F508del, and the other CFTR genetic mutation is R117H.

In some embodiments, the patient has at least one combination mutation chosen from:

### D443Y;G576A;R668C,

### F508C;S1251N,

### G576A; R668C,

### G970R; M470V,

### R74W;D1270N,

### R74W;V201M, and

### R74W;V201M;D1270N.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V and G1069R. In some embodiments, the patient possesses a CFTR genetic mutation selected from G178R, G551S, G970R, G1244E, S1255P, G1349D, S549N, S549R and S1251N. In some embodiments, the patient possesses a CFTR genetic mutation selected from E193K, F1052V and G1069R. In some embodiments, the method produces an increase in chloride transport relative to baseline chloride transport of the patient of the patient.

In some embodiments, in the methods of treating, lessening the severity of, or symptomatically treating cystic fibrosis disclosed herein, the patient possesses a CFTR genetic mutation selected from R117C, D110H, R347H, R352Q, E56K, P67L, L206W, A455E, D579G, S1235R, S945L, R1070W, F1074L, D110E, D1270N and D1152H.

In some embodiments, the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G. In some embodiments, the patient possesses a CFTR genetic mutation selected from 1717-1G->A, 1811+1.6kbA->G, 2789+5G->A, 3272-26A->G and 3849+10kbC->T. In some embodiments, the patient possesses a CFTR genetic mutation selected from 2789+5G->A and 3272-26A->G.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and human CFTR mutations selected from F508del, R117H, and G551D.

In some embodiments, in the methods of treating, lessening the severity of, or symptomatically treating cystic fibrosis disclosed herein, the patient possesses a CFTR genetic mutation selected from G178R, G551S, G970R, G1244E, S1255P, G1349D, S549N, S549R, S1251N, E193K, F1052V, G1069R, R117C, D110H, R347H, R352Q, E56K, P67L, L206W, A455E, D579G, S1235R, S945L, R1070W, F1074L, D110E, D1270N, D1152H, 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C, 621+3A->G, and a CFTR mutation selected from F508del, R117H, and G551D; and a CFTR mutations selected from F508del, R117H, and G551D.

In some embodiments, the patient possesses a CFTR genetic mutation selected from G178R, G551S, G970R, G1244E, S1255P, G1349D, S549N, S549R, S1251N, E193K, F1052V and G1069R, and a human CFTR mutation selected from F508del, R117H, and G551D. In some embodiments, the patient possesses a CFTR genetic mutation selected from G178R, G551S, G970R, G1244E, S1255P, G1349D, S549N, S549R and S1251N, and a human CFTR mutation selected from F508del, R117H, and G551D. In some embodiments, the patient possesses a CFTR genetic mutation selected from E193K, F1052V and G1069R, and a human CFTR mutation selected from F508del, R117H, and G551D.

In some embodiments, the patient possesses a CFTR genetic mutation selected from R117C, D110H, R347H, R352Q, E56K, P67L, L206W, A455E, D579G, S1235R, S945L, R1070W, F1074L, D110E, D1270N and D1152H, and a human CFTR mutation selected from F508del, R117H, and G551D.

In some embodiments, the patient possesses a CFTR genetic mutation selected from 1717-1G->A, 621+1G->T, 3120+1G->A, 1898+1G->A, 711+1G->T, 2622+1G->A, 405+1G->A, 406-1G->A, 4005+1G->A, 1812-1G->A, 1525-1G->A, 712-1G->T, 1248+1G->A, 1341+1G->A, 3121-1G->A, 4374+1G->T, 3850-1G->A, 2789+5G->A, 3849+10kbC->T, 3272-26A->G, 711+5G->A, 3120G->A, 1811+1.6kbA->G, 711+3A->G, 1898+3A->G, 1717-8G->A, 1342-2A->C, 405+3A->C, 1716G/A, 1811+1G->C, 1898+5G->T, 3850-3T->G, IVS14b+5G->A, 1898+1G->T, 4005+2T->C and 621+3A->G, and a human CFTR mutation selected from F508del, R117H, and G551D. In some embodiments, the patient possesses a CFTR genetic mutation selected from 1717-1G->A, 1811+1.6kbA->G, 2789+5G->A, 3272-26A->G and 3849+10kbC->T, and a human CFTR mutation selected from F508del, R117H, and G551D. In some embodiments, the patient possesses a CFTR genetic mutation selected from 2789+5G->A and 3272-26A->G, and a human CFTR mutation selected from F508del, R117H.

In some embodiments, the patient is heterozygous having a CF-causing mutation on one allele and a CF-causing mutation on the other allele. In some embodiments, the patient is heterozygous for F508del, and the other CFTR genetic mutation is any CF-causing mutation, including, but not limited to F508del on one CFTR allele and a CFTR mutation on the second CFTR allele that is associated with minimal CFTR function, residual CFTR function, or a defect in CFTR channel gating activity.

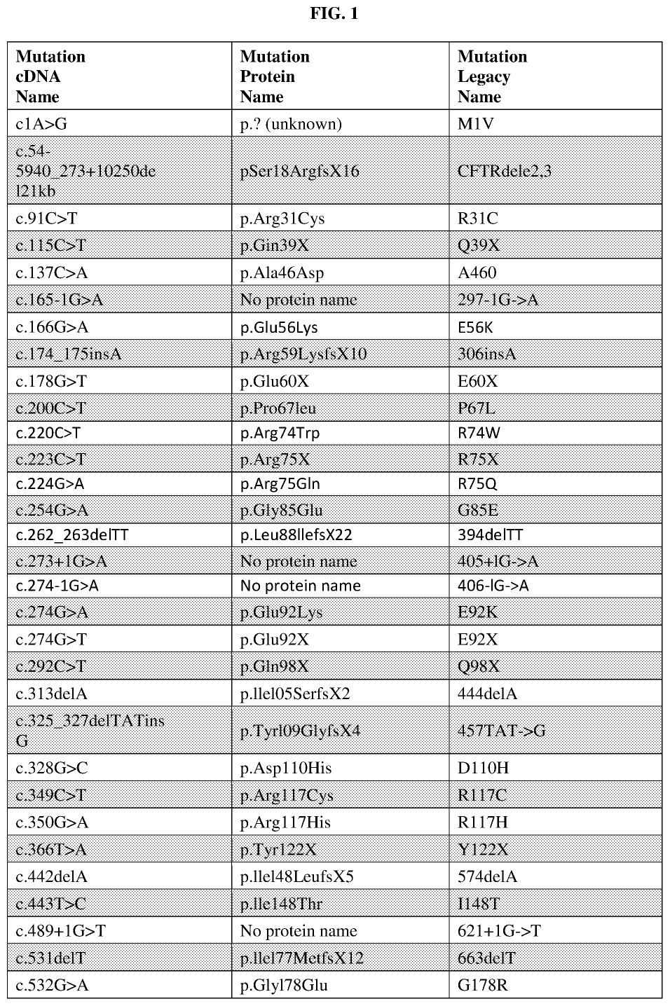

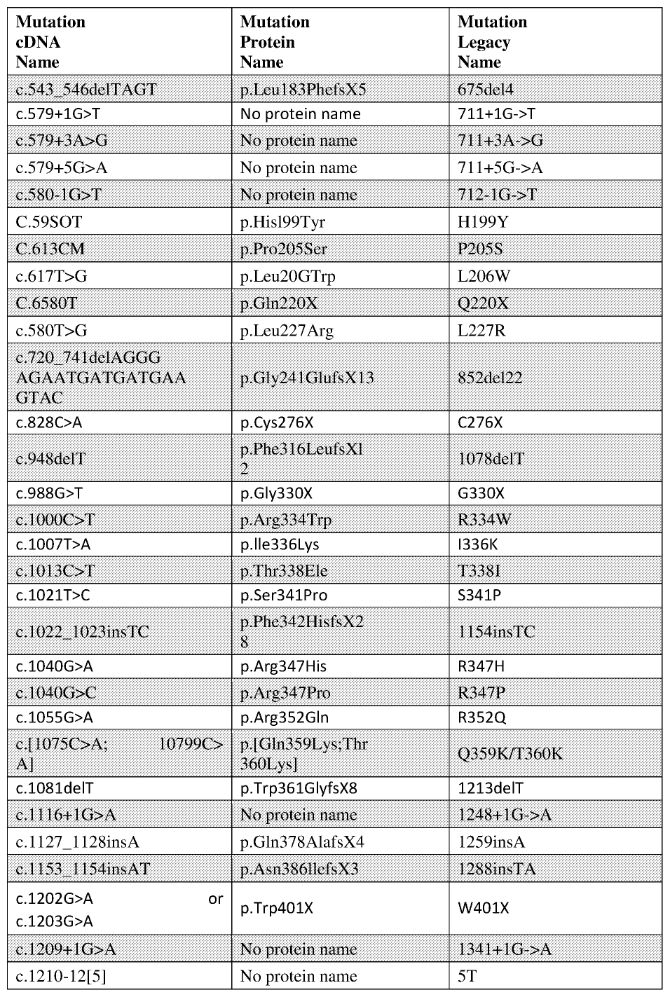

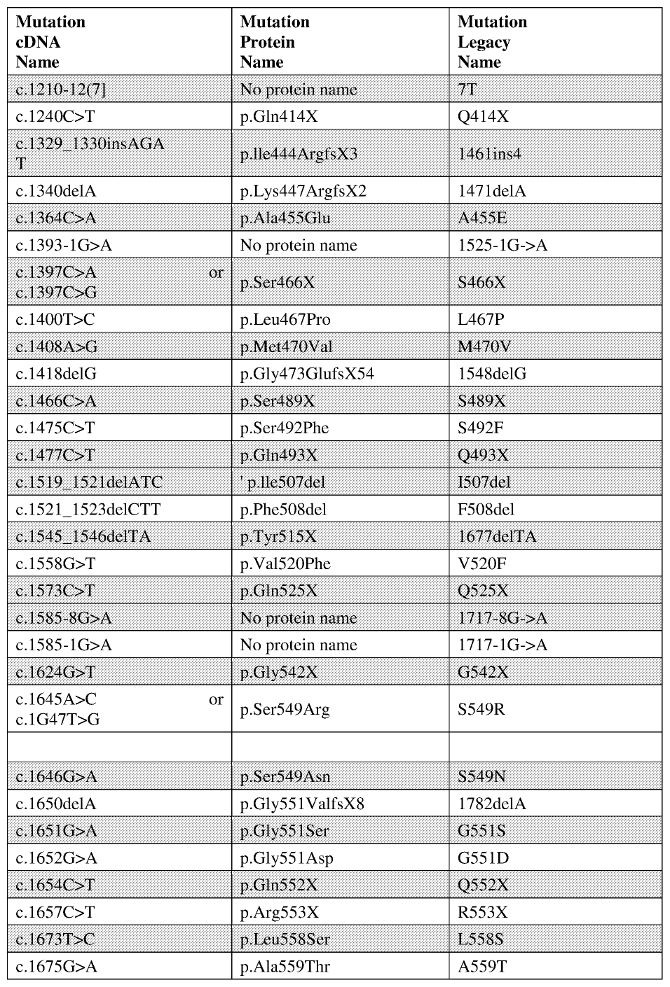

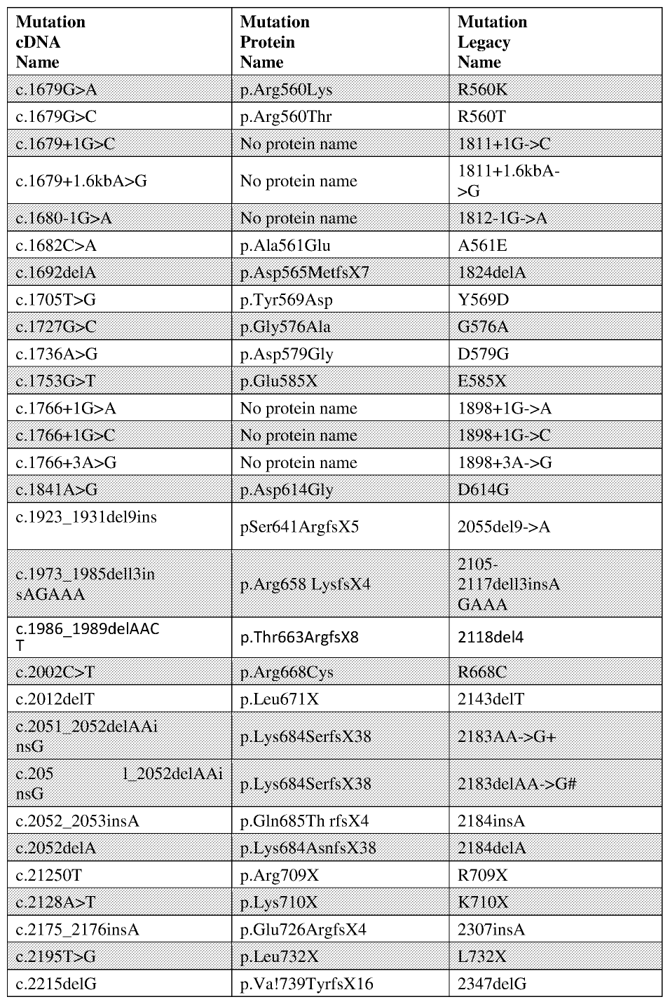

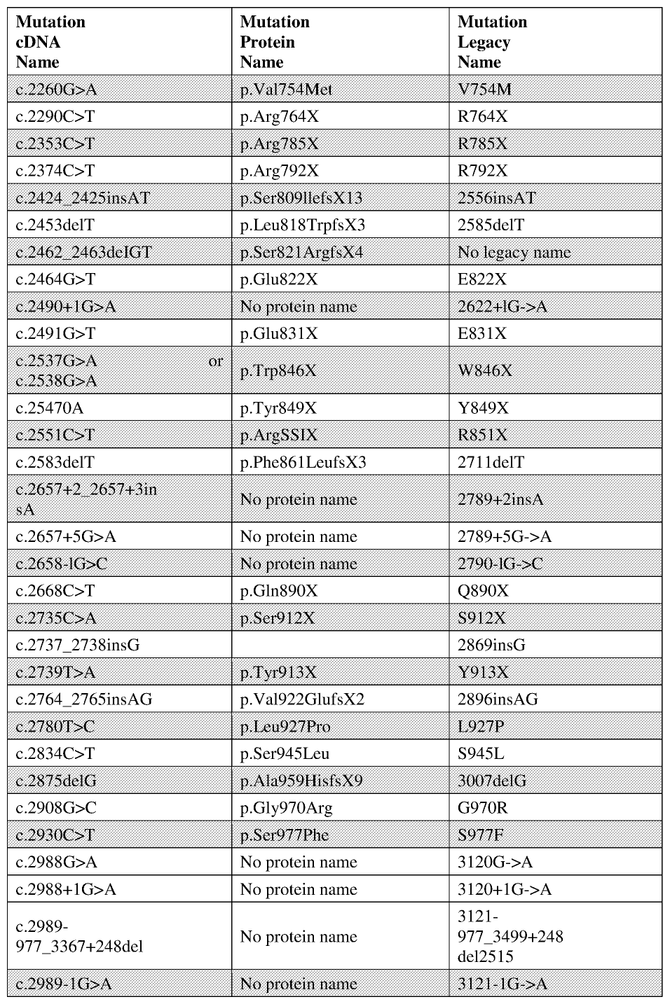

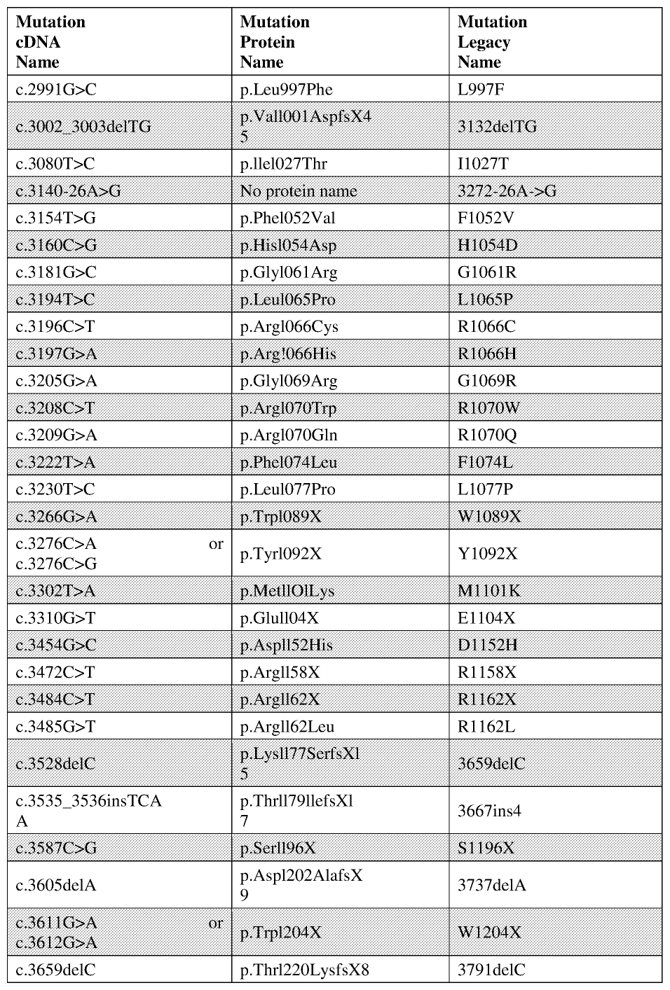

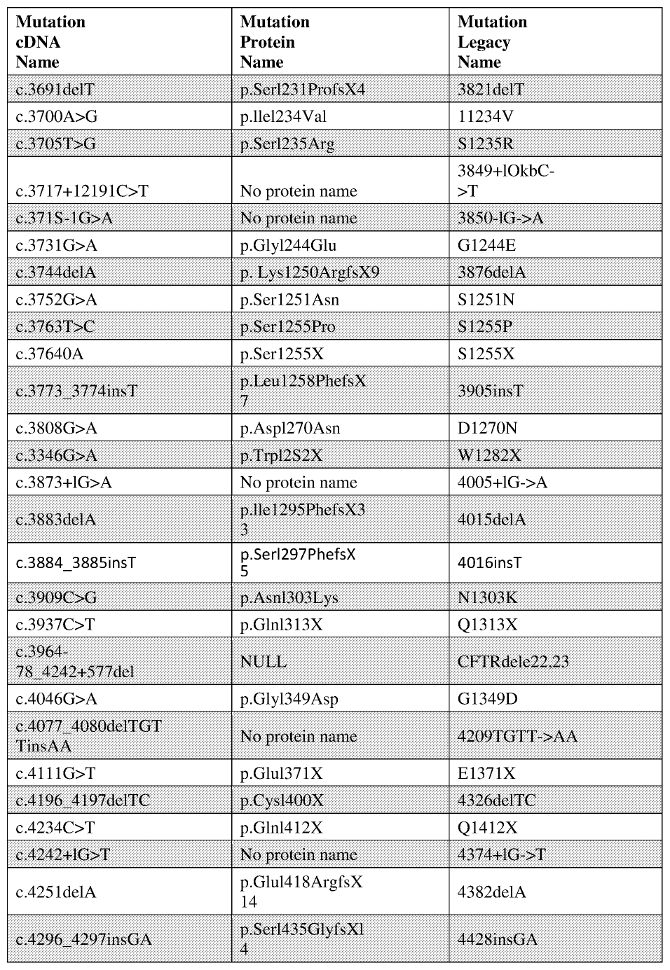

In some embodiments, the CF-causing mutation is selected from Table A. In some embodiments, the CF-causing mutation is selected from Table B. In some embodiments, the CF-causing mutation is selected from Table C. In some embodiments, the CF-causing mutation is selected from FIG. 1. In some embodiments, the patient is heterozygous having a CF-causing mutation on one CFTR allele selected from the mutations listed in the table from FIG. 1 and a CF-causing mutation on the other CFTR allele is selected from the CFTR mutations listed in Table B:

TABLE B
CFTR Mutations

| | | |
|---|---|---|
| Q39X | 1248+1G→A | R560S |
| W57X | 1341+1G→A | A561E |
| E60X | 1717−1G→A | Y569D |
| R75X | 1811+1.6kbA→G | L1065P |
| E92X | 1811+1G→C | R1066C |
| Q98X | 1812−1G→A | R1066M |
| Y122X | 1898+1G→A | L1077P |
| L218X | 2622+1G→A | H1085R |
| Q220X | 3120+1G→A | M1101K |
| C276X | 3120G→A | N1303K |
| Q290X | 3850−1G→A | 3849+10kbC→T |
| G330X | 4005+1G→A | 3272−26A→G |
| W401X | 4374+1G→T | 711+3A→G |
| Q414X | 663delT | E56K |
| S434X | 2183AA→G | P67L |
| S466X | CFTRdel2,3 | R74W |
| S489X | 3659delC | D110E |
| Q493X | 394delTT | D110H |
| W496X | 2184insA | R117C |
| Q525X | 3905insT | L206W |
| G542X | 2184delA | R347H |
| Q552X | 1078delT | R352Q |
| R553X | 1154insTC | A455E |
| E585X | 2183delAA→G | D579G |
| G673X | 2143delT | E831X |
| R709X | 1677delTA | S945L |
| K710X | 3876delA | S977F |
| L732X | 2307insA | F1052V |
| R764X | 4382delA | R1070W |
| R785X | 4016insT | F1074L |
| R792X | 2347delG | D1152H |
| E822X | 3007delG | D1270N |
| W846X | 574delA | G178R |
| R851X | 2711delT | S549N |
| Q890X | 3791delC | S549R |
| S912X | CFTRdele22-23 | G551D |
| W1089X | 457TAT→G | G551S |
| Y1092X | 2043delG | G1244E |
| E1104X | 2869insG | S1251N |
| R1158X | 3600+2insT | S1255P |
| R1162X | 3737delA | G1349D |
| S1196X | 4040delA | |
| W1204X | 541delC | |
| S1255X | A46D | |
| W1282X | T338I | |
| Q1313X | R347P | |
| 621+1G→T | L927P | |
| 711+1G→T | G85E | |
| 711+5G→A | S341P | |
| 712−1G→T | L467P | |
| 405+1G→A | I507del | |
| 405+3A→C | V520F | |
| 406−1G→A | A559T | |
| 621+1G→T | R560T | |

TABLE C
CFTR Mutations

| Criteria | Mutation | | | | |
|---|---|---|---|---|---|
| Truncation | Q2X | L218X | Q525X | R792X | E1104X |
| mutations | S4X | Q220X | G542X | E822X | W1145X |
| % PI > 50% | W19X | Y275X | G550X | W882X | R1158X |
| and/or | G27X | C276X | Q552X | W846X | R1162X |
| SwCl⁻ > 86 | Q39X | Q290X | R553X | Y849X | S1196X |
| mmol/L | W57X | G330X | E585X | R851X | W1204X |
| No full-length | E60X | W401X | G673X | Q890X | L1254X |
| protein | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Splice mutations | 185+1G→T | 711+5G→A | 1717−8G→A | 2622+1G→A | 3121−1G→A |
| % PI > 50% | 296+1G→A | 712−1G→T | 1717−1G→A | 2790−1G→C | 3500−2A→G |
| and/or | 296+1G→t | 1248+1G→A | 1811+1G→C | 3040G→C | 3600+2insT |
| SwCl⁻ > 86 | 405+1G→A | 1249−1G→A | 1811+1.6kbA→G | (G970R) | 3850−1G→A |
| mmol/L | 405+3A→C | 1341+1G→A | 1811+1643G→T | 3120G→A | 4005+1G→A |
| No or little | 406−1G→A | 1525−2A→G | 1812−1G→A | 3120+1G→A | 4374+1G→T |
| mature mRNA | 621+1G→T | 1525−1G→A | 1898+1G→A | 3121−2A→G | |
| | 711+1G→T | | 1898+1G→C | | |
| Small (≤3 | 182delT | 1078delT | 1677delTA | 2711delT | 3737delA |
| nucleotide) | 306insA | 1119delA | 1782delA | 2732insA | 3791delC |
| insertion/deletion | 306delTAGA | 1138insG | 1824delA | 2869insG | 3821delT |
| (ins/del) frameshift | 365−366insT | 1154insTC | 1833delT | 2896insAG | 3876delA |
| mutations | 394delTT | 1161delC | 2043delG | 2942insT | 3878delG |
| % PI > 50% | 442delA | 1213delT | 2143delT | 2957delT | 3905insT |
| and/or | 444delA | 1259insA | 2183AA→G ^{a} | 3007delG | 4016insT |
| SwCl⁻ > 86 | 457TAT→G | 1288insTA | 2184delA | 3028delA | 4021dupT |
| mmol/L | 541delC | 1343delG | 2184insA | 3171delC | 4022insT |
| Garbled and/or | 574delA | 1471delA | 2307insA | 3171insC | 4040delA |
| truncated | 663delT | 1497delGG | 2347delG | 3271delGG | 4279insA |
| protein | 849delG | 1548delG | 2585delT | 3349insT | 4326delTC |
| | 935delA | 1609del CA | 2594delGT | 3659delC | |
| Non-small (>3 | CFTRdele1 | CFTRdele16-17B | | 1461ins4 | |
| nucleotide) | CFTRdele2 | CFTRdele17A,17B | | 1924del7 | |
| insertion/deletion | CFTRdele2,3 | CFTRdele17A-18 | | 2055del9→A | |
| (ins/del) frameshift | CFTRdele2-4 | CFTRdele19 | | 2105- | |
| mutations | | | | 2117del13insAGAAA | |
| % PI > 50% and/or | CFTRdele3-10,14B-16 | CFTRdele19-21 | | 2372del8 | |
| SwCl⁻ > 86 | CFTRdele4-7 | CFTRdele21 | | 2721del11 | |
| mmol/L | CFTRdele4-11 | CFTRdele22-24 | | 2991del32 | |
| Garbled and/or | CFTR50kbdel | CFTRdele22, 23 | | 3121-977_3499+248del2515 | |
| truncated | CFTRdup6b-10 | 124del23bp | | 3667ins4 | |
| protein | CFTRdele11 | 602del14 | | 4010del4 | |
| | CFTRdele13,14a | 852del22 | | 4209TGTT→AA | |
| | CFTRdele14b-17b | 991del5 | | | |
| Class II, III, IV | A46D^{b} | V520F | Y569D^{b} | N1303K | |
| mutations not | G85E | A559T^{b} | L1065P | | |
| responsive to | R347P | R560T | R1066C | | |
| Compound II, | L467P^{b} | R560S | L1077P^{b} | | |
| Compound III | I507del | A561E | M1101K | | |
| or Compund II/ | | | | | |
| Compound III | | | | | |
| % PI > 50% and/or | | | | | |
| SwCl⁻ > 86 | | | | | |
| mmol/L | | | | | |
| AND | | | | | |
| Not responsive | | | | | |
| in vitro to | | | | | |
| Compound II, | | | | | |
| Compound III | | | | | |
| or Compund II/ | | | | | |
| Compound III | | | | | |

CFTR: cyctic fibrosis transmembrane conductance regulator; SwCl: sweat chloride
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 Feb. 2016.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.
^{a} Also known as 2183delAA→G.
^{b}Unpublished data.

In some embodiments, the patient is: with F508del/MF (F/MF) genotypes (heterozygous for F508del and an MF mutation not expected to respond to CFTR modulators, such as Compound III); with F508del/F508del (F/F) genotype (homozygous for F508del); and/or with F508del/gating (F/G) genotypes (heterozygous for F508del and a gating mutation known to be CFTR modulator-responsive (e.g., Compound III-responsive). In some embodiments, the patient with F508del/MF (F/MF) genotypes has a MF mutation that is not expected to respond to Compound II, Compound III, and both of Compound II and Compound III. In some embodiments, the patient with F508del/MF (F/MF) genotypes has any one of the MF mutations in Table C.

In some embodiments, the patient is heterozygous for F508del, and the other CFTR genetic mutation is any CF-causing mutation, including truncation mutations, splice mutations, small (≤3 nucleotide) insertion or deletion (ins/del) frameshift mutations; non-small (>3 nucleotide) insertion or deletion (ins/del) frameshift mutations; and Class II, III, IV mutations not responsive to Compound III alone or in combination with Compound II or Compound IV.

In some embodiments, the patient is heterozygous for F508del, and the other CFTR genetic mutation is a truncation mutation. In some specific embodiments, the truncation mutation is a truncation mutation listed in Table C.

In some embodiments, the patient is heterozygous for F508del, and the other CFTR genetic mutation is a splice mutation. In some specific embodiments, the splice mutation is a splice mutation listed in Table C.

In some embodiments, the patient is heterozygous for F508del, and the other CFTR genetic mutation is a small (≤3 nucleotide) insertion or deletion (ins/del) frameshift mutation. In some specific embodiments, the small (≤3 nucleotide) insertion or deletion (ins/del) frameshift mutation is a small (≤3 nucleotide) insertion or deletion (ins/del) frameshift mutation listed in Table C.

In some embodiments, the patient is heterozygous for F508del, and the other CFTR genetic mutation is any CF-causing mutation expected to be and/or is responsive to, based on in vitro and/or clinical data, any combination of Compounds (I), (II), (III), (III′), and pharmaceutically acceptable salts thereof, and their deuterated derivatives).

In some embodiments, the patient is heterozygous for F508del, and the other CFTR genetic mutation is any CF-causing mutation expected to be and/or is responsive, based on in vitro and/or clinical data, to the triple combination of Compounds (I), (II), (III), (III′), and pharmaceutically acceptable salts thereof, and their deuterated derivatives).

In some embodiments, the patient is heterozygous for F508del, and the other CFTR genetic mutation is a non-small (>3 nucleotide) insertion or deletion (ins/del) frameshift mutation. In some specific embodiments, the non-small (>3 nucleotide) insertion or deletion (ins/del) frameshift mutation is a non-small (>3 nucleotide) insertion or deletion (ins/del) frameshift mutation listed in Table C.

In some embodiments, the patient is heterozygous for F508del, and the other CFTR genetic mutation is a Class II, III, IV mutations not responsive to Compound III alone or in combination with Compound II or Compound IV. In some specific embodiments, the Class II, III, IV mutations not responsive to Compound III alone or in combination with Compound II or Compound IV is a Class II, III, IV mutations not responsive to Compound III alone or in combination with Compound II or Compound IV listed in Table C.

In some embodiments, the patient is heterozygous for F508del, and the other CFTR genetic mutation is any mutation listed in Table C.

In some embodiments, the patient is heterozygous for F508del, and the other CFTR genetic mutation is any mutation, but other than F508del, listed in Table A, B, C, and FIG. 1.

In some embodiments, the patient is heterozygous for F508del, and the other CFTR genetic mutation is any mutation listed in Table A. In some embodiments, the patient is heterozygous for F508del, and the other CFTR genetic mutation is any mutation listed in Table B. In some embodiments, the patient is heterozygous for F508del, and the other CFTR genetic mutation is any mutation listed in Table C. In some embodiments, the patient is heterozygous for F508del, and the other CFTR genetic mutation is any mutation listed in FIG. 1.

In some embodiments, the patient is homozygous for F508del.

In some embodiments, the patient is heterozygous having one CF-causing mutation on one CFTR allele selected from the mutations listed in the table from FIG. 1 and another CF-causing mutation on the other CFTR allele is selected from the CFTR mutations listed in Table C.

In some embodiments, the composition disclosed herein is useful for treating, lessening the severity of, or symptomatically treating cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary Cl⁻ concentrations, or ex vivo biochemical or histochemical techniques to monitor cell surface density. Using such methods, residual CFTR activity can be readily detected for patients that are heterozygous or homozygous for a variety of different mutations, including patients heterozygous for the most common mutation, F508del, as well as other mutations such as the G551D mutation, or the R117H mutation. In some embodiments, compositions disclosed herein are useful for treating, lessening the severity of, or symptomatically treating cystic fibrosis in patients who exhibit little to no residual CFTR activity. In some embodiments, compositions disclosed herein are useful for treating, lessening the severity of, or symptomatically treating cystic fibrosis in patients who exhibit little to no residual CFTR activity in the apical membrane of respiratory epithelia.

In some embodiments, the compositions disclosed herein are useful for treating or lessening the severity of cystic fibrosis in patients who exhibit residual CFTR activity using pharmacological methods. Such methods increase the amount of CFTR present at the cell surface, thereby inducing a hitherto absent CFTR activity in a patient or augmenting the existing level of residual CFTR activity in a patient.

In some embodiments, the compositions disclosed herein are useful for treating or lessening the severity of cystic fibrosis in patients with certain genotypes exhibiting residual CFTR activity.

In some embodiments, compositions disclosed herein are useful for treating, lessening the severity of, or symptomatically treating cystic fibrosis in patients within certain clinical phenotypes, e.g., a mild to moderate clinical phenotype that typically correlates with the amount of residual CFTR activity in the apical membrane of epithelia. Such phenotypes include patients exhibiting pancreatic sufficiency.

In some embodiments, the compositions disclosed herein are useful for treating, lessening the severity of, or symptomatically treating patients diagnosed with pancreatic sufficiency, idiopathic pancreatitis and congenital bilateral absence of the vas deferens, or mild lung disease wherein the patient exhibits residual CFTR activity.

In some embodiments, this disclosure relates to a method of augmenting or inducing anion channel activity in vitro or in vivo, comprising contacting the channel with a composition disclosed herein. In some embodiments, the anion channel is a chloride channel or a bicarbonate channel. In some embodiments, the anion channel is a chloride channel.

In some embodiments of the methods of treating cystic fibrosis disclosed herein, the absolute change in the patient's percent predicted forced expiratory volume in one second (ppFEV₁) after 15 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 40% relative to the ppFEV1 of the patient prior to said administration.

In some embodiments of the methods of treating cystic fibrosis disclosed herein, the absolute change in ppFEV₁ after 29 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 40% relative to the ppFEV1 of the patient prior to said administration. In some embodiments of the methods of treating cystic fibrosis disclosed herein, the absolute change in ppFEV₁ after 29 days ranges from 3% to 20% relative to the ppFEV1 of the patient prior to said administration.

In some embodiments of the methods of treating cystic fibrosis disclosed herein, the absolute change in the patient's sweat chloride after 15 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from −2 to −65 mmol/L from baseline, i.e., relative to the sweat chloride of the patient prior to said administration. In some embodiments, the absolute change in sweat chloride of said patient ranges from −5 to −65 mmol/L. In some embodiments, the absolute change in sweat chloride of said patient ranges from −10 to −65 mmol/L. In some embodiments, the absolute change in sweat chloride of said patient ranges from −10 to −45 mmol/L.

In some embodiments of the methods of treating cystic fibrosis disclosed herein, the absolute change in the patient's sweat chloride after 29 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from −2 to −65 mmol/L from baseline, i.e., relative to the sweat chloride of the patient prior to said administration. In some embodiments, the absolute change in sweat chloride of said patient ranges from −5 to −65 mmol/L. In some embodiments, the absolute change in sweat chloride of said patient ranges from −10 to −65 mmol/L. In some embodiments, the absolute change in sweat chloride of said patient ranges from −10 to −45 mmol/L. In some embodiments, the absolute change in sweat chloride of said patient ranges from −15 to −30 mmol/L.

In some embodiments, the triple combinations are administered to a patient who has one F508del mutation and one minimal function mutation, and who has not taken any of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof.

In some embodiments, the triple combinations are administered to a patient has two copies of F508del mutation, and wherein patient has taken at least one compound chosen from Compound II and pharmaceutically acceptable salts thereof, and at least one compound chosen from Compound III and pharmaceutically acceptable salts thereof, but not any of said at least one compound chosen from Compound I and pharmaceutically acceptable salts thereof.

In some embodiments, the absolute change in patient's ppFEV₁ after 15 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 35% relative to the ppFEV1 of the patient prior to said administration.

In some embodiments, the absolute change in patient's ppFEV₁ after 29 days of administration of at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 35% relative to the ppFEV1 of the patient prior to said administration.

In some embodiments, the absolute change in a patient's ppFEV1 relative to the ppFEV1 of the patient prior to such administration of the triple combinations can be calculated as (postbaseline value-baseline value). The baseline value is defined as the most recent non-missing measurement collected before the first dose of study drug in the Treatment Period (Day 1).

The exact amount of a pharmaceutical composition required will vary from subject to subject, depending on the species, age, and general condition of the subject, the severity of the disease, the particular agent, its mode of administration, and the like. The compounds of this disclosure may be formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, such as a mammal, and even further such as a human.

In some embodiments, the disclosure also is directed to methods of treatment using isotope-labelled embodiments of the afore-mentioned compounds I, II, and III, which, in some embodiments, are referred to as Compound I′, Compound II′, or Compound III′. In some embodiments, Compound I′, Compound II′, Compound III′, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The deuterium (²H)-labelled compounds and salts can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism.

In some embodiments, Compound III′ as used herein includes the deuterated compound disclosed in U.S. Pat. No. 8,865,902 (which is incorporated herein by reference), and CTP-656.

In some embodiments, Compound III′ is:

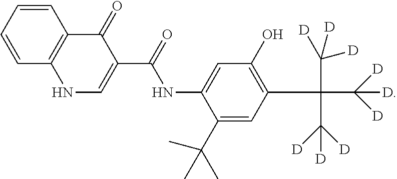

Exemplary embodiments of the disclosure include:

1. A method of treating cystic fibrosis comprising administering to a patient in need thereof:

(A) 50 mg to 1000 mg of at least one compound chosen from Compound I

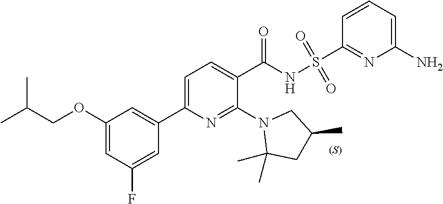

and pharmaceutically acceptable salts thereof daily; and

(B) 25 mg to 200 mg of at least one compound chosen from Compound II:

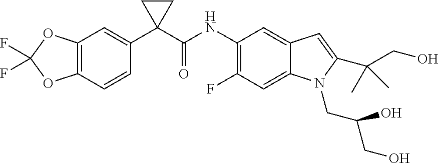 [SMILES: CC(C)(CO)c1cc2cc(NC(=O)C3(c4ccc5c(c4)OC(F)(F)O5)CC3)c(F)cc2n1C[C@@H](O)CO]

and pharmaceutically acceptable salts thereof daily; and

(C) 50 mg to 600 mg of at least one compound chosen from Compound III:

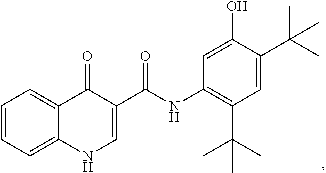 [SMILES: CC(C)(C)c1cc(C(C)(C)C)c(NC(=O)c2c[nH]c3ccccc3c2=O)cc1O]

and pharmaceutically acceptable salts thereof daily.
2. The method according to embodiment 1, wherein 100 mg to 800 mg, 100 mg to 700 mg, 200 mg to 700 mg, 200 mg to 600 mg, 300 mg to 600 mg, 400 mg to 600 mg, 500 mg to 700 mg, or 500 mg to 600 mg of at least one compound chosen from Compound I and pharmaceutically acceptable salts thereof is administered daily.
3. The method according to embodiment 1, wherein 100 mg of at least one compound chosen from Compound I and pharmaceutically acceptable salts thereof is administered daily.
4. The method according to embodiment 1, wherein 200 mg of at least one compound chosen from Compound I and pharmaceutically acceptable salts thereof is administered daily.
5. The method according to embodiment 1, wherein 300 mg of at least one compound chosen from Compound I and pharmaceutically acceptable salts thereof is administered daily.
6. The method according to embodiment 1, wherein 400 mg of at least one compound chosen from Compound I and pharmaceutically acceptable salts thereof is administered daily.
7. The method according to embodiment 1, wherein 500 mg of at least one compound chosen from Compound I and pharmaceutically acceptable salts thereof is administered daily.
8. The method according to embodiment 1, wherein 600 mg of at least one compound chosen from Compound I and pharmaceutically acceptable salts thereof is administered daily.
9. The method according to embodiment 1, wherein 700 mg of at least one compound chosen from Compound I and pharmaceutically acceptable salts thereof is administered daily.
10. The method according to embodiment 1, wherein 800 mg of at least one compound chosen from Compound I and pharmaceutically acceptable salts thereof is administered daily.
11. The method according to any one of embodiments 1-10, wherein at least one compound chosen from Compound I and pharmaceutically acceptable salts thereof is administered once daily.
12. The method according to any one of embodiments 1-10, wherein at least one compound chosen from Compound I and pharmaceutically acceptable salts thereof is administered twice daily.
13. The method according to any one of embodiments 1-12, wherein 50 mg to 150 mg or from 75 mg to 200 mg of at least one compound chosen from Compound II and pharmaceutically acceptable salts thereof is administered daily.
14. The method according to embodiment 13, wherein 50 mg of at least one compound chosen from Compound II and pharmaceutically acceptable salts thereof is administered daily.
15. The method according to embodiment 13, wherein 100 mg of at least one compound chosen from Compound II and pharmaceutically acceptable salts thereof is administered daily.
16. The method according to any one of embodiments 1-15, wherein at least one compound chosen from Compound II and pharmaceutically acceptable salts thereof is administered once daily.
17. The method according to any one of embodiments 1-15, wherein at least one compound chosen from Compound II and pharmaceutically acceptable salts thereof is administered in twice daily.
18. The method according to any one of embodiments 1-17, wherein 50 mg to 450 mg, from 100 mg to 400 mg, 125 mg to 300 mg, or 150 mg to 300 mg of at least one compound chosen from Compound III and pharmaceutically acceptable salts thereof is administered daily.
19. The method according to embodiment 18, wherein 150 mg of at least one compound chosen from Compound III and pharmaceutically acceptable salts thereof is administered daily.
20. The method according embodiment 18, wherein 300 mg of at least one compound chosen from Compound III and pharmaceutically acceptable salts thereof is administered daily.
21. The method according to any one of embodiments 1-20, wherein at least one compound chosen from Compound III and pharmaceutically acceptable salts thereof is administered once daily.
22. The method according to any one of embodiments 1-20, wherein the dose of at least one compound chosen from Compound III and pharmaceutically acceptable salts thereof is administered twice daily.
23. The method according to embodiment 1, wherein 100 mg to 600 mg of at least one compound chosen from Compound I and pharmaceutically acceptable salts thereof is administered daily; 100 mg of at least one compound chosen from Compound II and pharmaceutically acceptable salts thereof is administered once daily; and 150 mg or 300 mg of at least one compound chosen from Compound III and pharmaceutically acceptable salts thereof is administered twice daily.
24. The method according to embodiment 1, wherein 100 mg to 600 mg of at least one compound chosen from Compound I and pharmaceutically acceptable salts thereof is administered daily; 50 mg of at least one compound chosen from Compound II and pharmaceutically acceptable salts thereof is administered twice daily; and 150 mg or 300 mg of at least one compound chosen from Compound III and pharmaceutically acceptable salts thereof is administered twice daily.
25. The method according to embodiment 1, wherein 100 mg, 200 mg, or 300 mg of at least one compound chosen from Compound I and pharmaceutically acceptable salts thereof is administered twice daily; 100 mg of Compound II is administered once daily; and 150 mg or 300 mg of Compound III is administered twice daily.
26. The method according to embodiment 1, wherein 100 mg, 200 mg, or 300 mg of at least one compound chosen from Compound I and pharmaceutically acceptable salts thereof is administered twice daily; 50 mg of Compound II is administered twice daily; and 150 mg or 300 mg of Compound III is administered twice daily.
27. The method according to any one of embodiments 1-26, wherein said patient has cystic fibrosis is chosen from patients with F508del/minimal function genotypes, patients with F508del/F508del genotypes, patients with F508del/gating genotypes, patients with F508del/residual function genotypes, and patients with F508del/ another CFTR genetic mutation that is expected to be and/or is responsive to the triple combination of Compound I, Compound II, and/or Compound III genotypes based on in vitro and/or clinical data.
28. The method according to embodiment 27, wherein the patient with a F508del/minimal function genotype has a minimal function mutation selected from:

Mutation

| S4X | C276X | G542X | R792X | E1104X |
|---|---|---|---|---|
| G27X | Q290X | G550X | E822X | R1158X |
| Q39X | G330X | Q552X | W846X | R1162X |
| W57X | W401X | R553X | Y849X | S1196X |
| E60X | Q414X | E585X | R851X | W1204X |
| R75X | S434X | G673X | Q890X | L1254X |
| E92X | S466X | Q685X | S912X | S1255X |
| Q98X | S489X | R709X | Y913X | W1282X |
| Y122X | Q493X | K710X | W1089X | Q1313X |
| E193X | W496X | L732X | Y1092X | E1371X |
| L218X | C524X | R764X | W1098X | Q1382X |
| Q220X | Q525X | R785X | R1102X | Q1411X |
| 185+1G→T | 711+5G→A | 1717−8G→A | 2622+1G→A | 3121−1G→A |
| 296+1G→A | 712−1G→T | 1717−1G→A | 2790−1G→C | 3500−2A→G |
| 405+1G→A | 1248+1G→A | 1811+1G→C | 3040G→C | 3600+2insT |
| 405+3A→C | 1249−1G→A | 1811+1.6kbA→G | (G970R) | 3850−1G→A |
| 406−1G→A | 1341+1G→A | 1812−1G→A | 3120G→A | 4005+1G→A |
| 621+1G→T | 1525−2A→G | 1898+1G→A | 3120+1G→A | 4374+1G→T |
| 711+1G→T | 1525−1G→A | 1898+1G→C | 3121−2A→G | |
| 182delT | 1119delA | 1782delA | 2732insA | 3876delA |
| 306insA | 1138insG | 1824delA | 2869insG | 3878delG |
| 365-366insT | 1154insTC | 2043delG | 2896insAG | 3905insT |
| 394delTT | 1161delC | 2143delT | 2942insT | 4016insT |
| 442delA | 1213delT | 2183AA→G* | 2957delT | 4021dupT |
| 444delA | 1259insA | 2184delA | 3007delG | 4040delA |
| 457TAT→G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609del CA | 2594delGT | 3791delC | |
| 1078delT | 1677delTA | 2711delT | 3821delT | |
| CFTRdele2,3 | 1461ins4 | | 2991del32 | |
| CFTRdele22,23 | 1924del7 | | 3667ins4 | |
| 124del23bp | 2055del9→A | | 4010del4 | |
| 852del22 | 2105- | | 4209TGTT→AA | |
| | 2117del13insAGAAA | | | |
| 991del5 | 2721del11 | | | |
| A46D | V520F | Y569D^{b} | N1303K | |
| G85E | A559T^{b} | L1065P | | |
| R347P | R560T | R1066C | | |
| L467P | R560S | L1077P^{b} | | |
| I507del | A561E | M1101K | | |

29. The method according to embodiment 27, wherein the patient with a F508del/gating genotype has a gating mutation selected from G178R, S549N, S549R, G551D, G551S, G1244E, S1251N, S1255P, and G1349D.
30. The method according to embodiment 27, wherein the patient with a F508del/residual function genotype has a residual function mutation selected from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, K1060T, R117H, S1235R, I1027T, R668C, G576A, M470V, L997F, R75Q, R1070Q, R31C, D614G, G1069R, R1162L, E56K, A1067T, E193K, and K1060T.
31. The method according to any one of embodiments 1-30, wherein the absolute change in said patient's percent predicted forced expiratory volume in one second (ppFEV₁) after 15 days of administration of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 40% relative to the ppFEV1 of the patient prior to said administration.
32. The method according to embodiment 31, wherein said patient has one F508del mutation and one minimal function mutation, and wherein patient has not taken any of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof.
33. The method according to embodiment 31, wherein said patient has two copies of F508del mutation, and wherein patient has taken at least one compound chosen from Compound II and pharmaceutically acceptable salts thereof, and at least one compound chosen from Compound III and pharmaceutically acceptable salts thereof, but not any of said at least one compound chosen from Compound I and pharmaceutically acceptable salts thereof.
34. The method according to any one of embodiments 31-33, wherein said absolute change in said patient's ppFEV₁ ranges from 3% to 35%.
35. The method according to any one of embodiments 1-34, wherein said at least one compound chosen from Compound I and pharmaceutically acceptable salts thereof is comprised in a first pharmaceutical composition; said at least one compound chosen from Compound II and pharmaceutically acceptable salts thereof is comprised in a second pharmaceutical composition; and said at least one compound chosen from Compound III and pharmaceutically acceptable salts thereof is comprised in a third pharmaceutical composition.
36. The method according to any one of embodiments 1-34, wherein said at least one compound chosen from Compound I and pharmaceutically acceptable salts thereof is comprised in a first pharmaceutical composition; and said at least one compound chosen from Compound II and pharmaceutically acceptable salts thereof and said at least one compound chosen from Compound III and pharmaceutically acceptable salts thereof are comprised in a second pharmaceutical composition.
37. The method of embodiment 36, wherein said second pharmaceutical composition comprises 1 half of a daily dose of said at least one compound chosen from Compound III and pharmaceutically acceptable salts thereof, and the other half of said at least one compound chosen from Compound III and pharmaceutically acceptable salts thereof is administered to said patient in a third pharmaceutical composition.
38. The method according to any one of embodiments 1-34, wherein said at least one compound chosen from Compound I and pharmaceutically acceptable salts thereof is comprised in a first pharmaceutical composition; said at least one compound chosen from Compound II and pharmaceutically acceptable salts thereof is comprised in a second pharmaceutical composition; and said at least one compound chosen from Compound III and pharmaceutically acceptable salts thereof are comprised in the first pharmaceutical composition.
39. The method according to any one of embodiments 1-34, wherein said at least one compound chosen from Compound I and pharmaceutically acceptable salts thereof; said at least one compound chosen from Compound II and pharmaceutically acceptable salts thereof; and said at least one compound chosen from Compound III and pharmaceutically acceptable salts thereof are comprised in a first pharmaceutical composition.
40. The method according to embodiment 39, wherein the first pharmaceutical composition is administered to the patient twice daily.
41. A method of treating cystic fibrosis comprising administering to a patient in need thereof:
(A) 100 mg of at least one compound chosen from Compound I and pharmaceutically acceptable salts thereof twice daily:

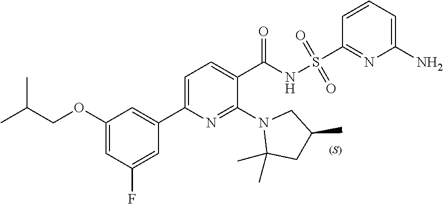

(B) 100 mg of at least one compound chosen from Compound II and pharmaceutically acceptable salts thereof once daily or 50 mg of at least one compound chosen from Compound II and pharmaceutically acceptable salts thereof twice daily:

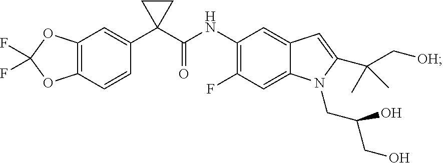 [SMILES: CC(C)(CO)c1cc2cc(NC(=O)C3(c4ccc5c(c4)OC(F)(F)O5)CC3)c(F)cc2n1C[C@@H](O)CO]

and
(C) 150 mg of at least one compound chosen from Compound III and pharmaceutically acceptable salts thereof twice daily:

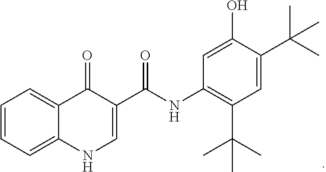 [SMILES: CC(C)(C)c1cc(C(C)(C)C)c(NC(=O)c2c[nH]c3ccccc3c2=O)cc1O]

42. A method of treating cystic fibrosis comprising administering to a patient in need thereof:
(A) 200 mg of at least one compound chosen from Compound I and pharmaceutically acceptable salts thereof twice daily:

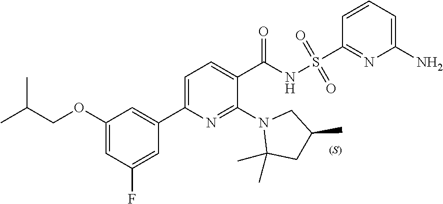

(B) 100 mg of at least one compound chosen from Compound II and pharmaceutically acceptable salts thereof once daily or 50 mg of at least one compound chosen from Compound II and pharmaceutically acceptable salts thereof twice daily:

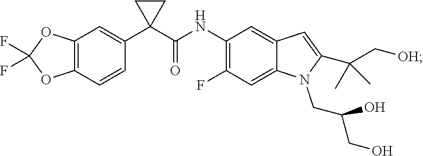 [SMILES: CC(C)(CO)c1cc2cc(NC(=O)C3(c4ccc5c(c4)OC(F)(F)O5)CC3)c(F)cc2n1C[C@@H](O)CO]

and
(C) 150 mg of at least one compound chosen from Compound III and pharmaceutically acceptable salts thereof twice daily:

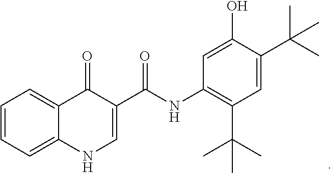 [SMILES: CC(C)(C)c1cc(C(C)(C)C)c(NC(=O)c2c[nH]c3ccccc3c2=O)cc1O]

43. A method of treating cystic fibrosis comprising administering to a patient in need thereof:
(A) 300 mg of at least one compound chosen from Compound I and pharmaceutically acceptable salts thereof twice daily:

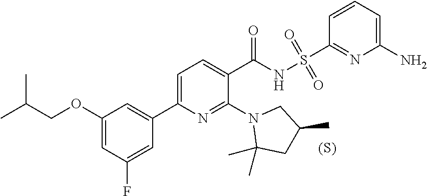

(B) 100 mg of at least one compound chosen from Compound II and pharmaceutically acceptable salts thereof once daily or 50 mg of at least one compound chosen from Compound II and pharmaceutically acceptable salts thereof twice daily:

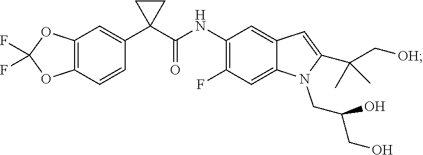 [SMILES: CC(C)(CO)c1cc2cc(NC(=O)C3(c4ccc5c(c4)OC(F)(F)O5)CC3)c(F)cc2n1C[C@@H](O)CO]

and
(C) 150 mg of at least one compound chosen from Compound III and pharmaceutically acceptable salts thereof twice daily:

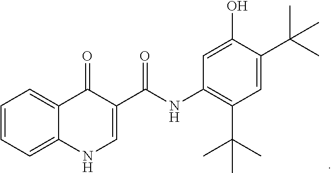 [SMILES: CC(C)(C)c1cc(C(C)(C)C)c(NC(=O)c2c[nH]c3ccccc3c2=O)cc1O]

44. A method of treating cystic fibrosis comprising administering to a patient in need thereof:
(A) 100 mg of at least one compound chosen from Compound I and pharmaceutically acceptable salts thereof twice daily:

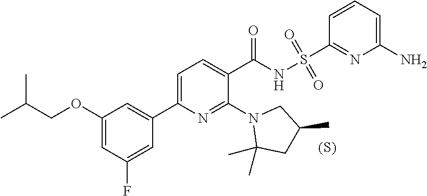

(B) 100 mg of at least one compound chosen from Compound II and pharmaceutically acceptable salts thereof once daily or 50 mg of at least one compound chosen from Compound II and pharmaceutically acceptable salts thereof twice daily:
and

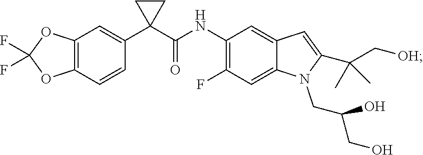 [SMILES: CC(C)(CO)c1cc2cc(NC(=O)C3(c4ccc5c(c4)OC(F)(F)O5)CC3)c(F)cc2n1C[C@@H](O)CO]

(C) 300 mg of at least one compound chosen from Compound III and pharmaceutically acceptable salts thereof twice daily:

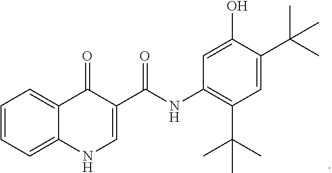 [SMILES: CC(C)(C)c1cc(C(C)(C)C)c(NC(=O)c2c[nH]c3ccccc3c2=O)cc1O]

45. A method of treating cystic fibrosis comprising administering to a patient in need thereof:
(A) 200 mg of at least one compound chosen from Compound I and pharmaceutically acceptable salts thereof twice daily:

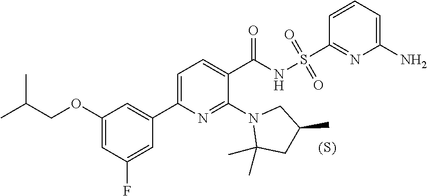

(B) 100 mg of at least one compound chosen from Compound II and pharmaceutically acceptable salts thereof once daily or 50 mg of at least one compound chosen from Compound II and pharmaceutically acceptable salts thereof twice daily:

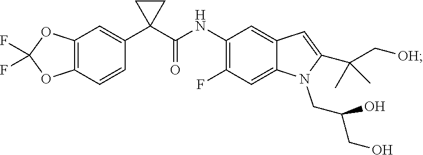 [SMILES: CC(C)(CO)c1cc2cc(NC(=O)C3(c4ccc5c(c4)OC(F)(F)O5)CC3)c(F)cc2n1C[C@@H](O)CO]

and
(C) 300 mg of at least one compound chosen from Compound III and pharmaceutically acceptable salts thereof twice daily:

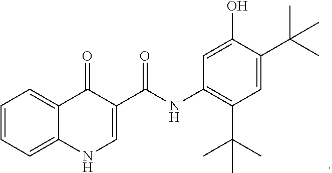 [SMILES: CC(C)(C)c1cc(C(C)(C)C)c(NC(=O)c2c[nH]c3ccccc3c2=O)cc1O]

46. A method of treating cystic fibrosis comprising administering to a patient in need thereof:
(A) 300 mg of at least one compound chosen from Compound I and pharmaceutically acceptable salts thereof twice daily:

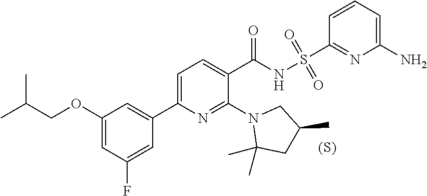

(B) 100 mg of at least one compound chosen from Compound II and pharmaceutically acceptable salts thereof once daily or 50 mg of at least one compound chosen from Compound II and pharmaceutically acceptable salts thereof twice daily:

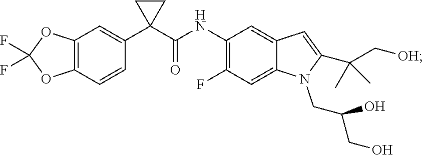 [SMILES: CC(C)(CO)c1cc2cc(NC(=O)C3(c4ccc5c(c4)OC(F)(F)O5)CC3)c(F)cc2n1C[C@@H](O)CO]

and
(C) 300 mg of at least one compound chosen from Compound III and pharmaceutically acceptable salts thereof twice daily:

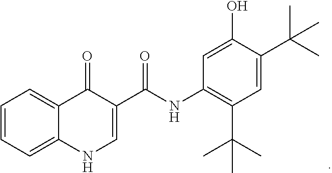 [SMILES: CC(C)(C)c1cc(C(C)(C)C)c(NC(=O)c2c[nH]c3ccccc3c2=O)cc1O]

47. The method according to any one of embodiments 40-45, wherein said patient has cystic fibrosis is chosen from patients with F508del/minimal function genotypes, patients with F508del/F508del genotypes, patients with F508del/gating genotypes, patients with F508del/residual function genotypes, and patients with F508del/ another CFTR genetic mutation that is expected to be and/or is responsive to the triple combination of Compound I, Compound II, and/or Compound III genotypes based on in vitro and/or clinical data.
48. The method according to embodiment 46, wherein the patient with a F508del/minimal function genotype has a minimal function mutation selected from:

Mutation

| | | | | |
|---|---|---|---|---|
| S4X | C276X | G542X | R792X | E1104X |
| G27X | Q290X | G550X | E822X | R1158X |
| Q39X | G330X | Q552X | W846X | R1162X |
| W57X | W401X | R553X | Y849X | S1196X |
| E60X | Q414X | E585X | R851X | W1204X |
| R75X | S434X | G673X | Q890X | L1254X |
| E92X | S466X | Q685X | S912X | S1255X |
| Q98X | S489X | R709X | Y913X | W1282X |
| Y122X | Q493X | K710X | W1089X | Q1313X |
| E193X | W496X | L732X | Y1092X | E1371X |
| L218X | C524X | R764X | W1098X | Q1382X |
| Q220X | Q525X | R785X | R1102X | Q1411X |
| 185+1G→T | 711+5G→A | 1717−8G→A | 2622+1G→A | 3121−1G→A |
| 296+1G→A | 712−1G→T | 1717−1G→A | 2790−1G→C | 3500−2A→G |
| 405+1G→A | 1248+1G→A | 1811+1G→C | 3040G→C | 3600+2insT |
| 405+3A→C | 1249−1G→A | 1811+1.6kbA→G | (G970R) | 3850−1G→A |
| 406−1G→A | 1341+1G→A | 1812−1G→A | 3120G→A | 4005+1G→A |
| 621+1G→T | 1525−2A→G | 1898+1G→A | 3120+1G→A | 4374+1G→T |
| 711+1G→T | 1525−1G→A | 1898+1G→C | 3121−2A→G | |
| 182delT | 1119delA | 1782delA | 2732insA | 3876delA |
| 306insA | 1138insG | 1824delA | 2869insG | 3878delG |
| 365-366insT | 1154insTC | 2043delG | 2896insAG | 3905insT |
| 394delTT | 1161delC | 2143delT | 2942insT | 4016insT |
| 442delA | 1213delT | 2183AA→G | 2957delT | 4021dupT |
| 444delA | 1259insA | 2184delA | 3007delG | 4040delA |
| 457TAT→G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609del CA | 2594delGT | 3791delC | |
| 1078delT | 1677delTA | 2711delT | 3821delT | |

| CFTRdele2, 3 | 1461ins4 | | 2991del32 |
|---|---|---|---|
| CFTRdele22, 23 | 1924del7 | | 3667ins4 |
| 124del23bp | 2055del9→A | | 4010del4 |
| 852del22 | 2105- | | 4209TGTT→AA |
| | 2117del13insAGAAA | | |
| 991del5 | 2721del11 | | |
| A46D^{b} | V520F | Y569D^{b} | N1303K |
| G85E | A559T^{b} | L1065P | |
| R347P | R560T | R1066C | |
| L467P^{b} | R560S | L1077P^{b} | |
| I507del | A561E | M1101K | |

49. The method according to embodiment 47, wherein the patient with a F508del/gating genotype has a gating mutation selected from G178R, S549N, S549R, G551D, G551S, G1244E, S1251N, S1255P, and G1349D.
50. The method according to embodiment 47, wherein the patient with a F508del/residual function genotype has a residual function mutation selected from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, K1060T, R117H, S1235R, I1027T, R668C, G576A, M470V, L997F, R75Q, R1070Q, R31C, D614G, G1069R, R1162L, E56K, A1067T, E193K, and K1060T.
51. The method according to any one of embodiments 41-50, wherein the absolute change in said patient's percent predicted forced expiratory volume in one second (ppFEV₁) after 15 days of administration of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 40% relative to the ppFEV1 of the patient prior to said administration.
52. The method according to embodiment 51, wherein said patient has one F508del mutation and one minimal function mutation, and wherein patient has not taken any of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof.
53. The method according to embodiment 51, wherein said patient has two copies of F508del mutation, and wherein patient has taken at least one compound chosen from Compound II and pharmaceutically acceptable salts thereof, and at least one compound chosen from Compound III and pharmaceutically acceptable salts thereof, but not any of said at least one compound chosen from Compound I and pharmaceutically acceptable salts thereof.
54. The method according to any one of embodiments 51-53, wherein said absolute change in said patient's ppFEV₁ ranges from 3% to 35%.
55. The method according to any one of embodiments 41-54, wherein said at least one compound chosen from Compound I and pharmaceutically acceptable salts thereof is comprised in a first pharmaceutical composition; said at least one compound chosen from Compound II and pharmaceutically acceptable salts thereof is comprised in a second pharmaceutical composition; and said at least one compound chosen from Compound III and pharmaceutically acceptable salts thereof is comprised in a third pharmaceutical composition.
56. The method according to any one of embodiments 41-54, wherein said at least one compound chosen from Compound I and pharmaceutically acceptable salts thereof is comprised in a first pharmaceutical composition; and said at least one compound chosen from Compound II and pharmaceutically acceptable salts thereof and said at least one compound chosen from Compound III and pharmaceutically acceptable salts thereof are comprised in a second pharmaceutical composition.
57. The method of embodiment 56, wherein said second pharmaceutical composition comprises a half of a daily dose of said at least one compound chosen from Compound III and pharmaceutically acceptable salts thereof, and the other half of said at least one compound chosen from Compound III and pharmaceutically acceptable salts thereof is administered to said patient in a third pharmaceutical composition.
58. The method according to any one of embodiments 41-54, wherein said at least one compound chosen from Compound I and pharmaceutically acceptable salts thereof is comprised in a first pharmaceutical composition; said at least one compound chosen from Compound II and pharmaceutically acceptable salts thereof is comprised in a second pharmaceutical composition; and said at least one compound chosen from Compound III and pharmaceutically acceptable salts thereof is comprised in the first pharmaceutical composition.
59. The method according to any one of embodiments 41-54, wherein said at least one compound chosen from Compound I and pharmaceutically acceptable salts thereof; said at least one compound chosen from Compound II and pharmaceutically acceptable salts thereof; and said at least one compound chosen from Compound III and pharmaceutically acceptable salts thereof are comprised in a first pharmaceutical composition.
60. The method according to embodiment 58, wherein the first pharmaceutical composition is administered to the patient twice daily.
61. A method of treating cystic fibrosis comprising administering to a patient in need thereof:
(A) 100 mg of Compound I twice daily:

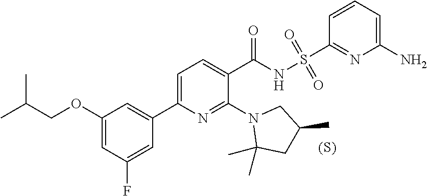

(B) 100 mg of Compound II once daily or 50 mg of Compound II twice daily:

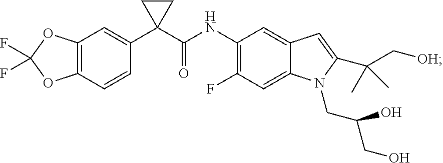 [SMILES: CC(C)(CO)c1cc2cc(NC(=O)C3(c4ccc5c(c4)OC(F)(F)O5)CC3)c(F)cc2n1C[C@@H](O)CO]

and
(C) 150 mg of Compound III twice daily:

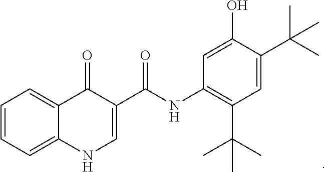 [SMILES: CC(C)(C)c1cc(C(C)(C)C)c(NC(=O)c2c[nH]c3ccccc3c2=O)cc1O]

62. A method of treating cystic fibrosis comprising administering to a patient in need thereof:
(A) 200 mg of Compound I twice daily:

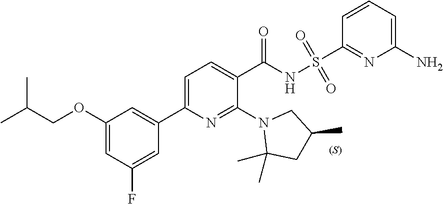

(B) 100 mg of Compound II once daily or 50 mg of Compound II twice daily:

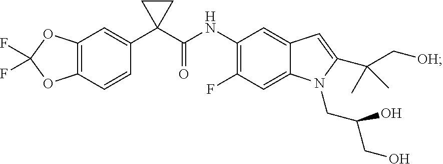 [SMILES: CC(C)(CO)c1cc2cc(NC(=O)C3(c4ccc5c(c4)OC(F)(F)O5)CC3)c(F)cc2n1C[C@@H](O)CO]

and
(C) 150 mg of Compound III twice daily:

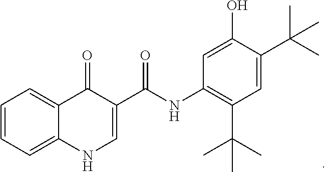 [SMILES: CC(C)(C)c1cc(C(C)(C)C)c(NC(=O)c2c[nH]c3ccccc3c2=O)cc1O]

63. A method of treating cystic fibrosis comprising administering to a patient in need thereof:
(A) 300 mg of Compound I twice daily:

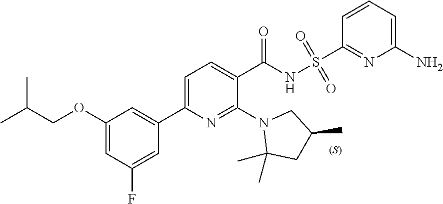

(B) 100 mg of Compound II once daily or 50 mg of Compound II twice daily:

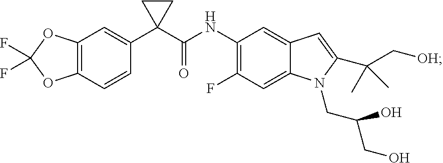 [SMILES: CC(C)(CO)c1cc2cc(NC(=O)C3(c4ccc5c(c4)OC(F)(F)O5)CC3)c(F)cc2n1C[C@@H](O)CO]

and
(C) 150 mg of Compound III twice daily:

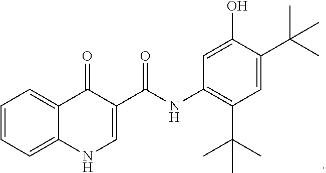 [SMILES: CC(C)(C)c1cc(C(C)(C)C)c(NC(=O)c2c[nH]c3ccccc3c2=O)cc1O]

64. A method of treating cystic fibrosis comprising administering to a patient in need thereof:
(A) 100 mg of Compound I twice daily:

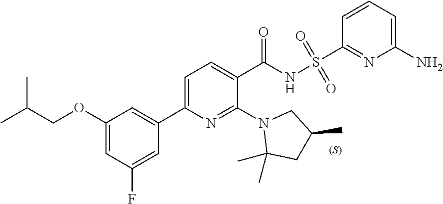

(B) 100 mg of Compound II once daily or 50 mg of Compound II twice daily:

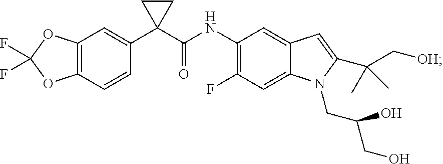 [SMILES: CC(C)(CO)c1cc2cc(NC(=O)C3(c4ccc5c(c4)OC(F)(F)O5)CC3)c(F)cc2n1C[C@@H](O)CO]

and
(C) 300 mg of Compound III twice daily:

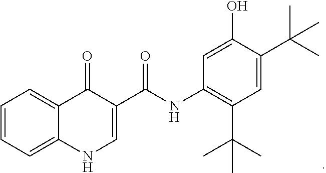 [SMILES: CC(C)(C)c1cc(C(C)(C)C)c(NC(=O)c2c[nH]c3ccccc3c2=O)cc1O]

65. A method of treating cystic fibrosis comprising administering to a patient in need thereof:
(A) 200 mg of Compound I twice daily:

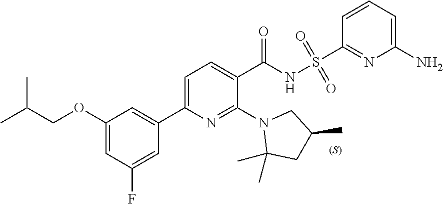

(B) 100 mg of Compound II once daily or 50 mg of Compound II twice daily:

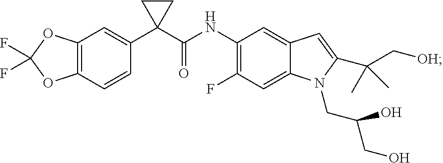 [SMILES: CC(C)(CO)c1cc2cc(NC(=O)C3(c4ccc5c(c4)OC(F)(F)O5)CC3)c(F)cc2n1C[C@@H](O)CO]

and
(C) 300 mg of Compound III twice daily:

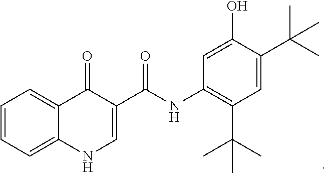 [SMILES: CC(C)(C)c1cc(C(C)(C)C)c(NC(=O)c2c[nH]c3ccccc3c2=O)cc1O]

66. A method of treating cystic fibrosis comprising administering to a patient in need thereof:
(A) 300 mg of Compound I twice daily:

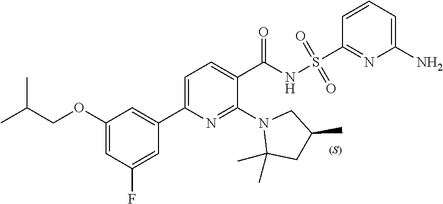

(B) 100 mg of Compound II once daily or 50 mg of Compound II twice daily:

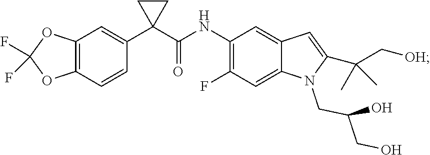 [SMILES: CC(C)(CO)c1cc2cc(NC(=O)C3(c4ccc5c(c4)OC(F)(F)O5)CC3)c(F)cc2n1C[C@@H](O)CO]

and
(C) 300 mg of Compound III twice daily:

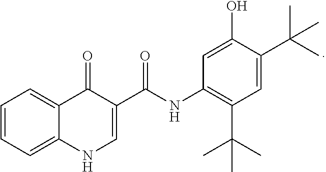 [SMILES: CC(C)(C)c1cc(C(C)(C)C)c(NC(=O)c2c[nH]c3ccccc3c2=O)cc1O]

67. The method according to any one of embodiments 61-66, wherein said patient has cystic fibrosis is chosen from patients with F508del/minimal function genotypes, patients with F508del/F508del genotypes, patients with F508del/gating genotypes, and patients with F508del/residual function genotypes.
68. The method according to embodiment 67, wherein the patient with a F508del/minimal function genotype has a minimal function mutation selected from:

Mutation

| | | | | |
|---|---|---|---|---|
| S4X | C276X | G542X | R792X | E1104X |
| G27X | Q290X | G550X | E822X | R1158X |
| Q39X | G330X | Q552X | W846X | R1162X |
| W57X | W401X | R553X | Y849X | S1196X |
| E60X | Q414X | E585X | R851X | W1204X |
| R75X | S434X | G673X | Q890X | L1254X |
| E92X | S466X | Q685X | S912X | S1255X |
| Q98X | S489X | R709X | Y913X | W1282X |
| Y122X | Q493X | K710X | W1089X | Q1313X |
| E193X | W496X | L732X | Y1092X | E1371X |
| L218X | C524X | R764X | W1098X | Q1382X |
| Q220X | Q525X | R785X | R1102X | Q1411X |
| 185+1G→T | 711+5G→A | 1717−8G→A | 2622+1G→A | 3121−1G→A |
| 296+1G→A | 712−1G→T | 1717−1G→A | 2790−1G→C | 3500−2A→G |
| 405+1G→A | 1248+1G→A | 1811+1G→C | 3040G→C | 3600+2insT |
| 405+3A→C | 1249−1G→A | 1811+1.6kbA→G | (G970R) | 3850−1G→A |
| 406−1G→A | 1341+1G→A | 1812−1G→A | 3120G→A | 4005+1G→A |
| 621+1G→T | 1525−2A→G | 1898+1G→A | 3120+1G→A | 4374+1G→T |
| 711+1G→T | 1525−1G→A | 1898+1G→C | 3121−2A→G | |
| 182delT | 1119delA | 1782delA | 2732insA | 3876delA |
| 306insA | 1138insG | 1824delA | 2869insG | 3878delG |
| 365-366insT | 1154insTC | 2043delG | 2896insAG | 3905insT |
| 394delTT | 1161delC | 2143delT | 2942insT | 4016insT |
| 442delA | 1213delT | 2183AA→G | 2957delT | 4021dupT |
| 444delA | 1259insA | 2184delA | 3007delG | 4040delA |
| 457TAT→G | 1288insTA | 2184insA | 3028delA | 4279insA |
| 541delC | 1471delA | 2307insA | 3171delC | 4326delTC |
| 574delA | 1497delGG | 2347delG | 3659delC | |
| 663delT | 1548delG | 2585delT | 3737delA | |
| 935delA | 1609del CA | 2594delGT | 3791delC | |
| 1078delT | 1677delTA | 2711delT | 3821delT | |

| CFTRdele2,3 | 1461ins4 | | 2991del32 |
|---|---|---|---|
| CFTRdele22,23 | 1924del7 | | 3667ins4 |
| 124del23bp | 2055del9→A | | 4010del4 |
| 852del22 | 2105- | | 4209TGTT→AA |
| | 2117del13insAGAAA | | |
| 991del5 | 2721del11 | | |
| A46D^{b} | V520F | Y569D^{b} | N1303K |
| G85E | A559T^{b} | L1065P | |
| R347P | R560T | R1066C | |
| L467P^{b} | R560S | L1077P^{b} | |
| I507del | A561E | M1101K | |

69. The method according to embodiment 67, wherein the patient with a F508del/gating genotype has a gating mutation selected from G178R, S549N, S549R, G551D, G551S, G1244E, S1251N, S1255P, and G1349D.
70. The method according to embodiment 67, wherein the patient with a F508del/residual function genotype has a residual function mutation selected from 2789+5G→A, 3849+10kbC→T, 3272-26A→G, 711+3A→G, E56K, P67L, R74W, D110E, D110H, R117C, L206W, R347H, R352Q, A455E, D579G, E831X, S945L, S977F, F1052V, R1070W, F1074L, D1152H, D1270N, E193K, K1060T, R117H, S1235R, I1027T, R668C, G576A, M470V, L997F, R75Q, R1070Q, R31C, D614G, G1069R, R1162L, E56K, A1067T, E193K, and K1060T.
71. The method according to any one of embodiments 61-70, wherein the absolute change in said patient's percent predicted forced expiratory volume in one second (ppFEV₁) after 15 days of administration of said Compound I, Compound II, and Compound III ranges from 3% to 40% relative to the ppFEV1 of the patient prior to said administration.
72. The method according to embodiment 71, wherein said patient has one F508del mutation and one minimal function mutation, and wherein patient has not taken any of said Compound I, Compound II, and Compound III.
73. The method according to embodiment 71, wherein said patient has two copies of F508del mutation, and wherein patient has taken Compound II and Compound III, but not said Compound I.
74. The method according to any one of embodiments 61-73, wherein said absolute change in said patient's ppFEV₁ ranges from 3% to 35%.
75. The method according to any one of embodiments 61-73, wherein Compound I is comprised in a first pharmaceutical composition; Compound II is comprised in a second pharmaceutical composition; and Compound III is comprised in a third pharmaceutical composition.
76. The method according to any one of embodiments 61-73, wherein Compound I is comprised in a first pharmaceutical composition; and Compound II and Compound III are comprised in a second pharmaceutical composition.
77. The method of embodiment 76, wherein said second pharmaceutical composition comprises one half of the daily dose of Compound III, and the other half of the daily dose of Compound III is administered to said patient in a third pharmaceutical composition.
78. The method according to any one of embodiments 61-73, wherein Compound I is comprised in a first pharmaceutical composition; Compound II is comprised in a second pharmaceutical composition; and Compound III is comprised in the first pharmaceutical composition.
79. The method according to any one of embodiments 61-73, wherein said Compound I, Compound II, and Compound III are comprised in a first pharmaceutical composition.
80. The method according to embodiment 79, wherein the first pharmaceutical composition is administered to the patient twice daily.
81. The method according to any one of embodiments 1-30 and 31, wherein the absolute change in said patient's ppFEV₁ after 29 days of administration of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 40% relative to the ppFEV1 of the patient prior to said administration.
82. The method according to any one of embodiments 31-33 and 81, wherein said absolute change in said patient's ppFEV₁ ranges from 3% to 35%.
83. The method according to any one of embodiment 41-50 and 51, wherein the absolute change in said patient's percent predicted forced expiratory volume in one second (ppFEV₁) after 15 days of administration of said at least one compound chosen from Compound I and pharmaceutically acceptable salts thereof, at least one compound chosen from Compound II and pharmaceutically acceptable salts thereof, and at least one compound chosen from Compound III and pharmaceutically acceptable salts thereof ranges from 3% to 40% relative to the ppFEV1 of the patient prior to said administration.
84. The method according to any one of embodiments 51-53 and 83, wherein said absolute change in said patient's ppFEV₁ ranges from 3% to 35%.
85. The method according to any one of embodiments 61-70 and 71, wherein the absolute change in said patient's percent predicted forced expiratory volume in one second (ppFEV₁) after 15 days of administration of said Compound I, Compound II, and Compound III ranges from 3% to 40% relative to the ppFEV1 of the patient prior to said administration.
86. The method according to any one of embodiments 61-73 and 85, wherein said absolute change in said patient's ppFEV₁ ranges from 3% to 35%.
87. The method according to any of the foregoing embodiments, wherein Compound III is replaced by Compound III′.
88. The method according to embodiment 87, wherein the daily dose of Compound III′ is 150 mg or 200 mg.

### EXAMPLES

### I. Methods of Preparing Compounds

General Experimental Procedures

Reagents and starting materials were obtained by commercial sources unless otherwise stated and were used without purification. Proton and carbon NMR spectra were acquired on either of a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. Proton and carbon spectra were either acquired with temperature control at 30° C. or ambient temperature using standard, previously published pulse sequences and routine processing parameters. Final purity of compounds was determined by reversed phase UPLC using an Acquity UPLC BEH C18 column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were obtained using a single quadrupole mass spectrometer with a mass accuracy of 0.1 Da and a minimum resolution of 1000 amu across the detection range using electrospray ionization (ESI) using the hydrogen ion (H⁺).

Compounds I, II and III can be prepared by any suitable method in the art, for example, PCT Publication Nos. WO 2011/133751 and WO 2015/160787.

### Example 1. Synthesis of Compound I: N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide

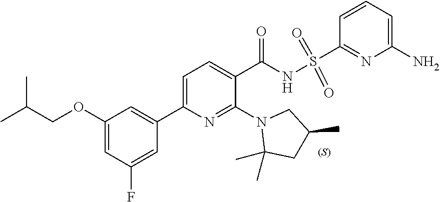

### Step 1: tert-butyl 2-chloro-6-(3-fluoro-5-isobutoxy-phenyl)pyridine-3-carboxylate

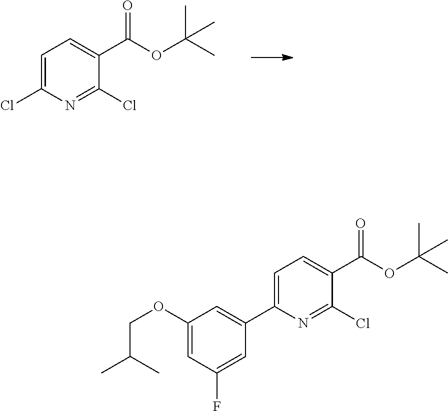 [SMILES: CC(C)(C)OC(=O)c1ccc(Cl)nc1Cl ; CC(C)COc1cc(F)cc(-c2ccc(C(=O)OC(C)(C)C)c(Cl)n2)c1]

tert-Butyl 2,6-dichloropyridine-3-carboxylate (15.0 g, 60.5 mmol) and (3-fluoro-5-isobutoxy-phenyl)boronic acid (13.46 g, 63.48 mmol) were combined and fully dissolved in ethanol (150 mL) and toluene (150 mL). A suspension of sodium carbonate (19.23 g, 181.4 mmol) in water (30 mL) was added. Tetrakis(triphenylphosphine)palladium (0) (2.096 g, 1.814 mmol) was added under nitrogen. The reaction mixture was allowed to stir at 60° C. for 16 hours. Volatiles were removed under reduced pressure. The remaining solids were partitioned between water (100 mL) and ethyl acetate (100 mL). The organic layer was washed with brine (lx 100 mL), dried over sodium sulfate, filtered, and concentrated under reduced pressure. The material was subjected silica gel column chromatography on a 330 gram silica gel column, 0 to 20% ethyl acetate in hexanes gradient. The material was repurified on a 220 gram silica gel column, isocratic 100% hexane for 10 minutes, then a 0 to 5% ethyl acetate in hexanes gradient to yield tert-butyl 2-chloro-6-(3-fluoro-5-isobutoxy-phenyl)pyridine-3-carboxylate (18.87 g, 49.68 mmol, 82.2%) as a colorless oil. ¹H NMR (400 MHz, DMSO-d₆) δ 8.24 (d, J=8.0 Hz, 1H), 8.16 (d, J=8.1 Hz, 1H), 7.48 (dd, J=9.4, 2.0 Hz, 2H), 6.99 (dt, J=10.8, 2.2 Hz, 1H), 3.86 (d, J=6.5 Hz, 2H), 2.05 (dt, J=13.3, 6.6 Hz, 1H), 1.57 (d, J=9.3 Hz, 9H), 1.00 (t, J=5.5 Hz, 6H). ESI-MS m/z calc. 379.13504, found 380.2 (M+1)⁺; Retention time: 2.57 minutes.

### Step 2: 2-chloro-6-(3-fluoro-5-isobutoxy-phenyl)pyridine-3-carboxylic acid

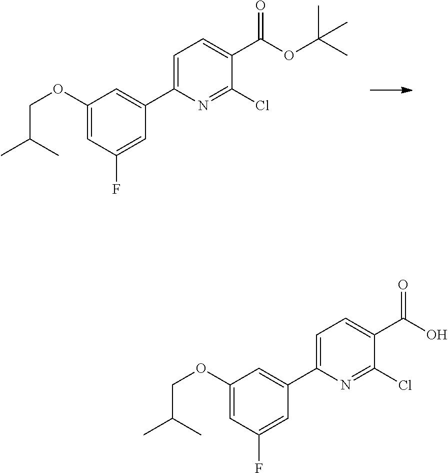

tert-Butyl 2-chloro-6-(3-fluoro-5-isobutoxy-phenyl)pyridine-3-carboxylate (18.57 g, 48.89 mmol) was dissolved in dichloromethane (200 mL). Trifluoroacetic acid (60 mL, 780 mmol) was added and the reaction mixture was allowed to stir at room temperature for 1 hour. The reaction mixture was stirred at 40° C. for 2 hours. The reaction mixture was concentrated under reduced pressure and taken up in ethyl acetate (100 mL). It was washed with a saturated aqueous sodium bicarbonate solution (lx 100 mL) and brine (1×100 mL), dried over sodium sulfate, filtered, and concentrated under reduced pressure. The crude product was suspended in ethyl acetate (75 mL) and washed with aqueous HCl (1 N, lx 75 mL). The organic layer was dried over sodium sulfate, filtered and concentrated under reduced pressure. The remaining solid (17.7 g) was stirred as a slurry in dichloromethane (35 mL) at 40° C. for 30 minutes. After cooling to room temperature, the remaining slurry was filtered, and then rinsed with cold dichloromethane to give 2-chloro-6-(3-fluoro-5-isobutoxy-phenyl)pyridine-3-carboxylic acid (11.35 g, 35.06 mmol, 72%) as a white solid. ¹H NMR (400 MHz, DMSO-d₆) δ 13.76 (s, 1H), 8.31 (d, J=8.0 Hz, 1H), 8.17 (d, J=8.1 Hz, 1H), 7.54-7.47 (m, 2H), 7.00 (dt, J=10.8, 2.3 Hz, 1H), 3.87 (d, J=6.5 Hz, 2H), 2.05 (dt, J=13.3, 6.6 Hz, 1H), 1.01 (d, J=6.7 Hz, 6H). ESI-MS m/z calc. 323.1, found 324.1 (M+1)⁺; Retention time: 1.96 minutes.

### Step 3: N-[(6-amino-2-pyridyl)sulfonyl]-2-chloro-6-(3-fluoro-5-isobutoxy-phenyl)pyridine-3-carboxamide

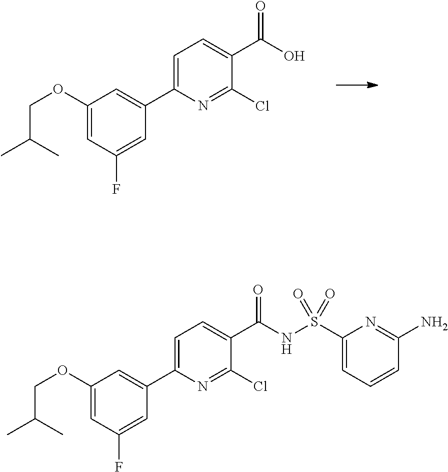 [SMILES: CC(C)COc1cc(F)cc(-c2ccc(C(=O)O)c(Cl)n2)c1 ; CC(C)COc1cc(F)cc(-c2ccc(C(=O)NS(=O)(=O)c3cccc(N)n3)c(Cl)n2)c1]

2-Chloro-6-(3-fluoro-5-isobutoxy-phenyl)pyridine-3-carboxylic acid (3.00 g, 9.27 mmol) was dissolved in N,N-dimethylformamide (30.00 mL), and 1,1′-carbonyldiimidazole (2.254 g, 13.90 mmol) was added to the solution. The solution was allowed to stir at 65° C. for 1 hour. In a separate flask, sodium hydride (444.8 mg, 11.12 mmol) was added to a solution of 6-aminopyridine-2-sulfonamide (1.926 g, 11.12 mmol) in N,N-dimethylformamide (15.00 mL). This mixture was stirred for one hour before being added to the prior reaction mixture. The final reaction mixture was stirred at 65° C. for 15 minutes. Volatiles were removed under reduced pressure. The remaining oil was taken up in ethyl acetate and washed with aqueous HCl (1 N, lx 75 mL) and brine (3×75 mL). The organic layer was dried over sodium sulfate, filtered, and concentrated under reduced pressure. The remaining white solid (4.7 g) was fully dissolved in isopropanol (120 mL) in an 85° C. water bath. The colorless solution was allowed to slowly cool to room temperature with slow stirring over 16 hours. The crystalline solids that had formed were collected by vacuum filtration, and then rinsed with cold isopropanol (50 mL). Upon drying, N-[(6-amino-2-pyridyl)sulfonyl]-2-chloro-6-(3-fluoro-5-isobutoxy-phenyl)pyridine-3-carboxamide (3.24 g, 6.765 mmol, 73%) was obtained as a white solid. ¹H NMR (400 MHz, DMSO-d₆) δ 12.78 (s, 1H), 8.15 (d, J=8.0 Hz, 1H), 8.09 (d, J=7.9 Hz, 1H), 7.73-7.63 (m, 1H), 7.49 (dd, J=8.6, 1.9 Hz, 2H), 7.21 (d, J=7.3 Hz, 1H), 6.99 (dt, J=10.7, 2.2 Hz, 1H), 6.74 (d, J=8.4 Hz, 1H), 6.64 (s, 2H), 3.86 (d, J=6.5 Hz, 2H), 2.05 (dp, J=13.3, 6.5 Hz, 1H), 1.02 (dd, J=12.7, 6.4 Hz, 6H).

### Step 4: N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I) and N-[(6-amino-2-pyridyl)sulfonyl]-6-(3-fluoro-5-isobutoxy-phenyl)-2-[(4R)-2,2,4-trimethylpyrrolidin-1-yl]pyridine-3-carboxamide

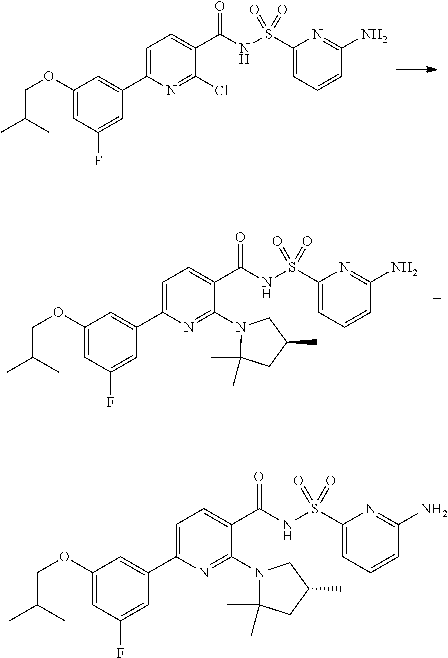 [SMILES: CC(C)COc1cc(F)cc(-c2ccc(C(=O)NS(=O)(=O)c3cccc(N)n3)c(Cl)n2)c1 ; CC(C)COc1cc(F)cc(-c2ccc(C(=O)NS(=O)(=O)c3cccc(N)n3)c(N3C[C@@H](C)CC3(C)C)n2)c1 ; CC(C)COc1cc(F)cc(-c2ccc(C(=O)NS(=O)(=O)c3cccc(N)n3)c(N3C[C@H](C)CC3(C)C)n2)c1]

N-[(6-Amino-2-pyridyl)sulfonyl]-2-chloro-6-(3-fluoro-5-isobutoxy-phenyl)pyridine-3-carboxamide (309 mg, 0.645 mmol) was dissolved in dimethylsulfoxide (3.708 mL) and potassium carbonate (445.9 mg, 3.226 mmol) was slowly added, followed by 2,2,4-trimethylpyrrolidine (146.0 mg, 1.290 mmol). The reaction mixture was sealed and heated at 150° C. for 72 hours. The reaction was cooled down, diluted with water (50 mL), extracted 3 times with 50 mL portions of ethyl acetate, washed with brine, dried over sodium sulfate, filtered and evaporated to dryness. The crude material was dissolved in 2 mL of dichloromethane and purified by on silica gel using a gradient of 0 to 80% ethyl acetate in hexanes. The stereoisomers were separated using supercritical fluid chromatography on a ChiralPak AD-H (250×4.6 mm), 5 μm column using 25% isopropanol with 1.0% diethylamine in CO₂ at a flow rate of 3.0 mL/min. The separated enationmers were separately concentrated, diluted with ethyl acetate (3 mL) and washed with 1N aqueous hydrochloric acid. The organic layers were dried over sodium sulfate, filtered, and evaporated to dryness to give the pure compounds as pale yellow solids.

The first compound to elute from the SFC conditions given above gave N-[(6-amino-2-pyridyl) sulfonyl]-6-(3-fluoro-5-isobutoxy-phenyl)-2-[(4R)-2,2,4-trimethylpyrrolidin-1-yl]pyridine-3-carboxamide (Hydrochloric Acid)¹H NMR (400 MHz, DMSO-d6) δ 12.47 (s, 1H), 7.78 (d, J=8.0 Hz, 1H), 7.69-7.57 (m, 1H), 7.56-7.46 (m, 1H), 7.41 (dt, J=10.1, 1.8 Hz, 1H), 7.26 (d, J=8.0 Hz, 1H), 7.21 (d, J=7.2 Hz, 1H), 6.89 (dt, J=10.7, 2.3 Hz, 1H), 6.69 (d, J=8.3 Hz, 1H), 3.83 (d, J=6.7 Hz, 2H), 2.61 (dq, J=9.7, 4.9 Hz, 2H), 2.24 (d, J=15.8 Hz, 1H), 2.06 (dq, J=13.3, 6.7 Hz, 1H), 1.93-1.82 (m, 1H), 1.61 (s, 3H), 1.59 (s, 3H), 1.48-1.33 (m, 1H), 1.32-1.20 (m, 2H), 0.99 (d, J=6.6 Hz, 6H), 0.88 (d, J=6.2 Hz, 3H). ESI-MS m/z calc. 555.2, found 556.4 (M+1)⁺; Retention time: 2.76 minutes.

The second compound to elute from the SFC conditions described above gave N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound I) (Hydrochloric Acid (1)) ¹H NMR (400 MHz, Chloroform-d) δ 15.49 (s, 1H), 8.49 (d, J=8.2 Hz, 1H), 7.75-7.56 (m, 3H), 7.34 (t, J=1.8 Hz, 1H), 7.30 (dt, J=9.4, 1.9 Hz, 1H), 6.75-6.66 (m, 2H), 3.95 (s, 1H), 3.78 (d, J=6.5 Hz, 2H), 3.42 (s, 1H), 2.88-2.74 (m, 1H), 2.23 (dd, J=12.5, 8.0 Hz, 1H), 2.17-2.08 (m, 1H), 1.98-1.87 (m, 1H), 1.55 (s, 3H), 1.39 (s, 3H), 1.31 (d, J=6.7 Hz, 3H), 1.05 (d, J=6.7 Hz, 6H). ESI-MS m/z calc. 555.2, found 556.4 (M+1)⁺; Retention time: 2.77 minutes. Absolute stereochemistry was confirmed by X-ray crystallography.

### Example 2. Synthesis of Compound II: (R)-1-(2,2-Difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

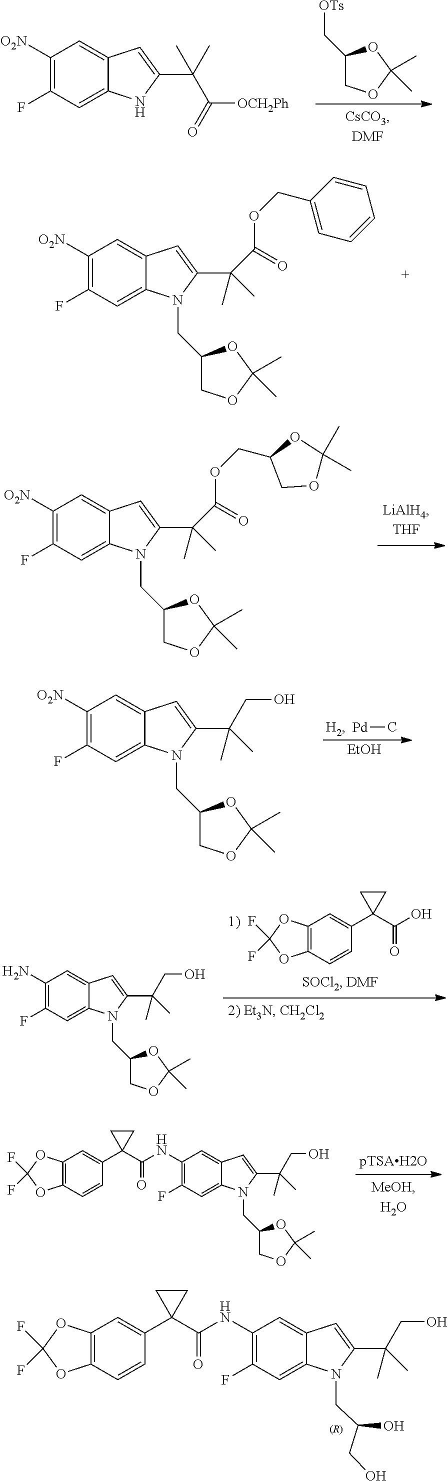

### Step A: (R)-Benzyl 2-(1-((2,2-dimethyl-1,3-dioxolan-4-yl)methyl)-6-fluoro-5-nitro-1H-indol-2-yl)-2-methylpropanoate and ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate

Cesium carbonate (8.23 g, 25.3 mmol) was added to a mixture of benzyl 2-(6-fluoro-5-nitro-1H-indol-2-yl)-2-methylpropanoate (3.0 g, 8.4 mmol) and (S)-(2,2-dimethyl-1,3-dioxolan-4-yl)methyl 4-methylbenzenesulfonate (7.23 g, 25.3 mmol) in DMF (17 mL). The reaction was stirred at 80° C. for 46 hours under a nitrogen atmosphere. The mixture was then partitioned between ethyl acetate and water. The aqueous layer was extracted with ethyl acetate. The combined ethyl acetate layers were washed with brine, dried over MgSO₄, filtered and concentrated. The crude product, a viscous brown oil which contains both of the products shown above, was taken directly to the next step without further purification. (R)-Benzyl 2-(1-((2,2-dimethyl-1,3-dioxolan-4-yl)methyl)-6-fluoro-5-nitro-1H-indol-2-yl)-2-methylpropanoate, ESI-MS m/z calc. 470.2, found 471.5 (M+1)⁺. Retention time 2.20 minutes. ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate, ESI-MS m/z calc. 494.5, found 495.7 (M+1)⁺. Retention time 2.01 minutes.

### Step B: (R)-2-(1-((2,2-dimethyl-1,3-dioxolan-4-yl)methyl)-6-fluoro-5-nitro-1H-indol-2-yl)-2-methylpropan-1-ol

The crude reaction mixture obtained in step (A) was dissolved in THF (42 mL) and cooled in an ice-water bath. LiAlH₄ (16.8 mL of 1 M solution, 16.8 mmol) was added drop-wise. After the addition was complete, the mixture was stirred for an additional 5 minutes. The reaction was quenched by adding water (1 mL), 15% NaOH solution (1 mL) and then water (3 mL). The mixture was filtered over Celite, and the solids were washed with THF and ethyl acetate. The filtrate was concentrated and purified by column chromatography (30-60% ethyl acetate-hexanes) to obtain (R)-2-(1-((2,2-dimethyl-1,3-dioxolan-4-yl)methyl)-6-fluoro-5-nitro-1H-indol-2-yl)-2-methylpropan-1-ol as a brown oil (2.68 g, 87% over 2 steps). ESI-MS m/z calc. 366.4, found 367.3 (M+1)⁺. Retention time 1.68 minutes. ¹H NMR (400 MHz, DMSO-d6) δ 8.34 (d, J=7.6 Hz, 1H), 7.65 (d, J=13.4 Hz, 1H), 6.57 (s, 1H), 4.94 (t, J=5.4 Hz, 1H), 4.64-4.60 (m, 1H), 4.52-4.42 (m, 2H), 4.16-4.14 (m, 1H), 3.76-3.74 (m, 1H), 3.63-3.53 (m, 2H), 1.42 (s, 3H), 1.38-1.36 (m, 6H) and 1.19 (s, 3H) ppm

### Step C: (R)-2-(5-amino-1-((2,2-dimethyl-1,3-dioxolan-4-yl)methyl)-6-fluoro-1H-indol-2-yl)-2-methylpropan-1-ol

(R)-2-(1-((2,2-dimethyl-1,3-dioxolan-4-yl)methyl)-6-fluoro-5-nitro-1H-indol-2-yl)-2-methylpropan-1-ol (2.5 g, 6.82 mmol) was dissolved in ethanol (70 mL) and the reaction was flushed with N2. Then Pd-C (250 mg, 5% wt) was added. The reaction was flushed with nitrogen again and then stirred under H₂ (atm). After 2.5 hours only partial conversion to the product was observed by LCMS. The reaction was filtered through Celite and concentrated. The residue was re-subjected to the conditions above. After 2 hours LCMS indicated complete conversion to product. The reaction mixture was filtered through Celite. The filtrate was concentrated to yield the product as a black solid (1.82 g, 79%). ESI-MS m/z calc. 336.2, found 337.5 (M+1)⁺. Retention time 0.86 minutes. ¹H NMR (400 MHz, DMSO-d6) δ 7.17 (d, J=12.6 Hz, 1H), 6.76 (d, J=9.0 Hz, 1H), 6.03 (s, 1H), 4.79-4.76 (m, 1H), 4.46 (s, 2H), 4.37-4.31 (m, 3H), 4.06 (dd, J=6.1, 8.3 Hz, 1H), 3.70-3.67 (m, 1H), 3.55-3.52 (m, 2H), 1.41 (s, 3H), 1.32 (s, 6H) and 1.21 (s, 3H) ppm.

### Step D: (R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide

DMF (3 drops) was added to a stirring mixture of 1-(2,2-difluorobenzo[d][1,3]dioxol-5-yl)cyclopropanecarboxylic acid (1.87 g, 7.7 mmol) and thionyl chloride (1.30 mL, 17.9 mmol). After 1 hour a clear solution had formed. The solution was concentrated under vacuum and then toluene (3 mL) was added and the mixture was concentrated again. The toluene step was repeated once more and the residue was placed on high vacuum for 10 minutes. The acid chloride was then dissolved in dichloromethane (10 mL) and added to a mixture of (R)-2-(5-amino-1-((2,2-dimethyl-1,3-dioxolan-4-yl)methyl)-6-fluoro-H-indol-2-yl)-2-methylpropan-1-ol (1.8 g, 5.4 mmol) and triethylamine (2.24 mL, 16.1 mmol) in dichloromethane (45 mL). The reaction was stirred at room temperature for 1 hour. The reaction was washed with 1N HCl solution, saturated NaHCO₃ solution and brine, dried over MgSO₄ and concentrated to yield the product as a black foamy solid (3 g, 100%). ESI-MS m/z calc. 560.6, found 561.7 (M+1)⁺. Retention time 2.05 minutes. ¹H NMR (400 MHz, DMSO-d6) δ 8.31 (s, 1H), 7.53 (s, 1H), 7.42-7.40 (m, 2H), 7.34-7.30 (m, 3H), 6.24 (s, 1H), 4.51-4.48 (m, 1H), 4.39-4.34 (m, 2H), 4.08 (dd, J=6.0, 8.3 Hz, 1H), 3.69 (t, J=7.6 Hz, 1H), 3.58-3.51 (m, 2H), 1.48-1.45 (m, 2H), 1.39 (s, 3H), 1.34-1.33 (m, 6H), 1.18 (s, 3H) and 1.14-1.12 (m, 2H) ppm

### Step E: (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

(R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide (3.0 g, 5.4 mmol) was dissolved in methanol (52 mL). Water (5.2 mL) was added followed by p-TsOH.H₂O (204 mg, 1.1 mmol). The reaction was heated at 80° C. for 45 minutes. The solution was concentrated and then partitioned between ethyl acetate and saturated NaHCO₃ solution. The ethyl acetate layer was dried over MgSO₄ and concentrated. The residue was purified by column chromatography (50-100% ethyl acetate-hexanes) to yield the product as a cream colored foamy solid. (1.3 g, 47%, ee>98% by SFC). ESI-MS m/z calc. 520.5, found 521.7 (M+1)⁺. Retention time 1.69 minutes. ¹H NMR (400 MHz, DMSO-d6) δ 8.31 (s, 1H), 7.53 (s, 1H), 7.42-7.38 (m, 2H), 7.33-7.30 (m, 2H), 6.22 (s, 1H), 5.01 (d, J=5.2 Hz, 1H), 4.90 (t, J=5.5 Hz, 1H), 4.75 (t, J=5.8 Hz, 1H), 4.40 (dd, J=2.6, 15.1 Hz, 1H), 4.10 (dd, J=8.7, 15.1 Hz, 1H), 3.90 (s, 1H), 3.65-3.54 (m, 2H), 3.48-3.33 (m, 2H), 1.48-1.45 (m, 2H), 1.35 (s, 3H), 1.32 (s, 3H) and 1.14-1.11 (m, 2H) ppm.

### Example 3. Synthesis of Compound III: N-(2,4-di-tert-butyl-5-hydroxyphenyl)-4-oxo-1,4-dihydroquinoline-3-carboxamide

### Part A: Preparation of 4-oxo-1,4-dihydroquinoline-3-carboxylic acid

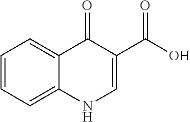 [SMILES: O=C(O)c1c[nH]c2ccccc2c1=O]

### Step A: 2-Phenylaminomethylene-malonic acid diethyl ester

A mixture of aniline (25.6 g, 0.275 mol) and diethyl 2-(ethoxymethylene)malonate (62.4 g, 0.288 mol) was heated at 140-150° C. for 2 h. The mixture was cooled to room temperature and dried under reduced pressure to afford 2-phenylaminomethylene-malonic acid diethyl ester as a solid, which was used in the next step without further purification. ¹H NMR (DMSO-d6) δ 11.00 (d, 1H), 8.54 (d, J=13.6 Hz, 1H), 7.36-7.39 (m, 2H), 7.13-7.17 (m, 3H), 4.17-4.33 (m, 4H), 1.18-1.40 (m, 6H).

### Step B: 4-Hydroxyquinoline-3-carboxylic acid ethyl ester

A 1 L three-necked flask fitted with a mechanical stirrer was charged with 2-phenylaminomethylene-malonic acid diethyl ester (26.3 g, 0.100 mol), polyphosphoric acid (270 g) and phosphoryl chloride (750 g). The mixture was heated to 70° C. and stirred for 4 h. The mixture was cooled to room temperature and filtered. The residue was treated with aqueous Na₂CO₃ solution, filtered, washed with water and dried. 4-Hydroxyquinoline-3-carboxylic acid ethyl ester was obtained as a pale brown solid (15.2 g, 70%). The crude product was used in next step without further purification.

### Step C: 4-Oxo-1,4-dihydroquinoline-3-carboxylic acid

4-Hydroxyquinoline-3-carboxylic acid ethyl ester (15 g, 69 mmol) was suspended in sodium hydroxide solution (2N, 150 mL) and stirred for 2 h at reflux. After cooling, the mixture was filtered, and the filtrate was acidified to pH 4 with 2N HCl. The resulting precipitate was collected via filtration, washed with water and dried under vacuum to give 4-oxo-1,4-dihydroquinoline-3-carboxylic acid as a pale white solid (10.5 g, 92%). ¹H NMR (DMSO-d6) δ 15.34 (s, 1H), 13.42 (s, 1H), 8.89 (s, 1H), 8.28 (d, J=8.0 Hz, 1H), 7.88 (m, 1H), 7.81 (d, J=8.4 Hz, 1H), 7.60 (m, 1H).

### Part B: N-(2,4-di-tert-butyl-5-hydroxyphenyl)-4-oxo-1,4-dihydroquinoline-3-carboxamide

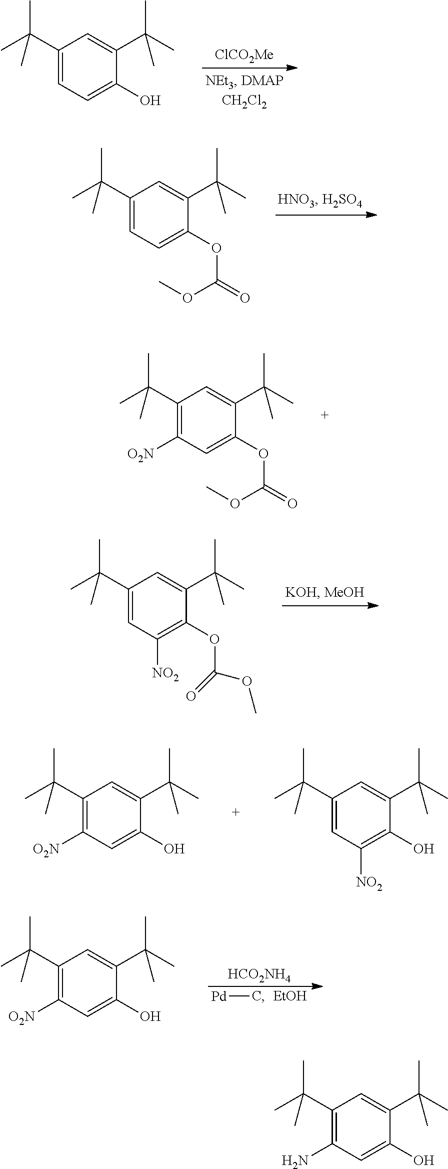

### Step A: Carbonic acid 2,4-di-tert-butyl-phenyl ester methyl ester

Methyl chloroformate (58 mL, 750 mmol) was added dropwise to a solution of 2,4-di-tert-butyl-phenol (103.2 g, 500 mmol), Et₃N (139 mL, 1000 mmol) and DMAP (3.05 g, 25 mmol) in dichloromethane (400 mL) cooled in an ice-water bath to 0° C. The mixture was allowed to warm to room temperature while stirring overnight, then filtered through silica gel (approx. 1 L) using 10% ethyl acetate-hexanes (˜4 L) as the eluent. The combined filtrates were concentrated to yield carbonic acid 2,4-di-tert-butyl-phenyl ester methyl ester as a yellow oil (132 g, quant.). ¹H NMR (400 MHz, DMSO-d6) δ 7.35 (d, J=2.4 Hz, 1H), 7.29 (dd, J=8.5, 2.4 Hz, 1H), 7.06 (d, J=8.4 Hz, 1H), 3.85 (s, 3H), 1.30 (s, 9H), 1.29 (s, 9H).

### Step B: Carbonic acid 2,4-di-tert-butyl-5-nitro-phenyl ester methyl ester and Carbonic acid 2,4-di-tert-butyl-6-nitro-phenyl ester methyl ester

To a stirring mixture of carbonic acid 2,4-di-tert-butyl-phenyl ester methyl ester (4.76 g, 180 mmol) in conc. sulfuric acid (2 mL), cooled in an ice-water bath, was added a cooled mixture of sulfuric acid (2 mL) and nitric acid (2 mL). The addition was done slowly so that the reaction temperature did not exceed 50° C. The reaction was allowed to stir for 2 h while warming to room temperature. The reaction mixture was then added to ice-water and extracted into diethyl ether. The ether layer was dried (MgSO₄), concentrated and purified by column chromatography (0-10% ethyl acetate-hexanes) to yield a mixture of carbonic acid 2,4-di-tert-butyl-5-nitro-phenyl ester methyl ester and carbonic acid 2,4-di-tert-butyl-6-nitro-phenyl ester methyl ester as a pale yellow solid (4.28 g), which was used directly in the next step.

### Step C: 2,4-Di-tert-butyl-5-nitro-phenol and 2,4-Di-tert-butyl-6-nitro-phenol

The mixture of carbonic acid 2,4-di-tert-butyl-5-nitro-phenyl ester methyl ester and carbonic acid 2,4-di-tert-butyl-6-nitro-phenyl ester methyl ester (4.2 g, 14.0 mmol) was dissolved in MeOH (65 mL) before KOH (2.0 g, 36 mmol) was added. The mixture was stirred at room temperature for 2 h. The reaction mixture was then made acidic (pH 2-3) by adding conc. HCl and partitioned between water and diethyl ether. The ether layer was dried (MgSO₄), concentrated and purified by column chromatography (0-5% ethyl acetate-hexanes) to provide 2,4-di-tert-butyl-5-nitro-phenol (1.31 g, 29% over 2 steps) and 2,4-di-tert-butyl-6-nitro-phenol. 2,4-Di-tert-butyl-5-nitro-phenol: ¹H NMR (400 MHz, DMSO-d6) δ 10.14 (s, 1H, OH), 7.34 (s, 1H), 6.83 (s, 1H), 1.36 (s, 9H), 1.30 (s, 9H). 2,4-Di-tert-butyl-6-nitro-phenol: ¹H NMR (400 MHz, CDCl₃) δ 11.48 (s, 1H), 7.98 (d, J=2.5 Hz, 1H), 7.66 (d, J=2.4 Hz, 1H), 1.47 (s, 9H), 1.34 (s, 9H).

### Step D: 5-Amino-2,4-di-tert-butyl-phenol

To a refluxing solution of 2,4-di-tert-butyl-5-nitro-phenol (1.86 g, 7.40 mmol) and ammonium formate (1.86 g) in ethanol (75 mL) was added Pd-5% wt. on activated carbon (900 mg). The reaction mixture was stirred at reflux for 2 h, cooled to room temperature and filtered through Celite. The Celite was washed with methanol and the combined filtrates were concentrated to yield 5-amino-2,4-di-tert-butyl-phenol as a grey solid (1.66 g, quant.). ¹H NMR (400 MHz, DMSO-d6) δ 8.64 (s, 1H, OH), 6.84 (s, 1H), 6.08 (s, 1H), 4.39 (s, 2H, NH₂), 1.27 (m, 18H); HPLC ret. time 2.72 min, 10-99% CH₃CN, 5 min run; ESI-MS 222.4 m/z [M+H]⁺.

### Step E: N-(5-hydroxy-2,4-di-tert-butyl-phenyl)-4-oxo-1H-quinoline-3-carboxamide

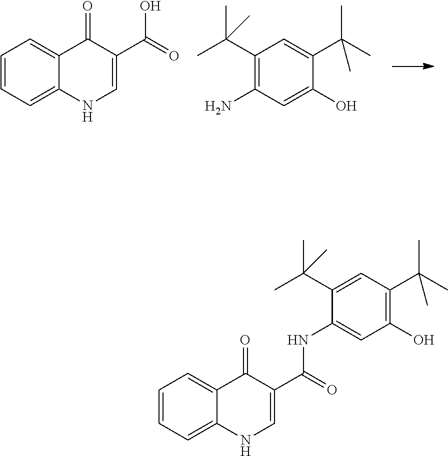

To a suspension of 4-oxo-1,4-dihydroquinolin-3-carboxylic acid (35.5 g, 188 mmol) and HBTU (85.7 g, 226 mmol) in DMF (280 mL) was added Et₃N (63.0 mL, 451 mmol) at ambient temperature. The mixture became homogeneous and was allowed to stir for 10 min before 5-amino-2,4-di-tert-butyl-phenol (50.0 g, 226 mmol) was added in small portions. The mixture was allowed to stir overnight at ambient temperature. The mixture became heterogeneous over the course of the reaction. After all of the acid was consumed (LC-MS analysis, MH+190, 1.71 min), the solvent was removed in vacuo. EtOH was added to the orange solid material to produce a slurry. The mixture was stirred on a rotovap (bath temperature 65° C.) for 15 min without placing the system under vacuum. The mixture was filtered and the captured solid was washed with hexanes to provide a white solid that was the EtOH crystalate. Et₂O was added to the solid obtained above until a slurry was formed. The mixture was stirred on a rotovap (bath temperature 25° C.) for 15 min without placing the system under vacuum. The mixture was filtered and the solid captured. This procedure was performed a total of five times. The solid obtained after the fifth precipitation was placed under vacuum overnight to provide N-(5-hydroxy-2,4-di-tert-butyl-phenyl)-4-oxo-1H-quinoline-3-carboxamide as a white powdery solid (38 g, 52%). HPLC ret. time 3.45 min, 10-99% CH₃CN, 5 min run; 1H NMR (400 MHz, DMSO-d6) δ 12.88 (s, 1H), 11.83 (s, 1H), 9.20 (s, 1H), 8.87 (s, 1H), 8.33 (dd, J=8.2, 1.0 Hz, 1H), 7.83-7.79 (m, 1H), 7.76 (d, J=7.7 Hz, 1H), 7.54-7.50 (m, 1H), 7.17 (s, 1H), 7.10 (s, 1H), 1.38 (s, 9H), 1.37 (s, 9H); ESI-MS m/z calc'd 392.21; found 393.3 [M+H]⁺.

### Example 4: Studies to Evaluate the Safety, Tolerability, and Bioavailability of Compound I

A randomized, double-blind, placebo-controlled, single- and multiple-dose, dose-escalation study was conduced in healthy volunteer subjects. Subjects were randomized to receive Compound I or placebo (Part A and Part B), and triple combination of Compound I, Compound II, and Compound III, or triple placebo (Part C).

In summary, Compound I was well tolerated as single doses from 50 mg up to 2000 mg and as multiple doses up to 400 mg q12h for 14 days and up to 300 mg q12h in triple combination with Compound II (100 mg qd) and Compound III (150 mg q12h) for 13 days. Dose-limiting adverse events were observed with multiple doses of 800 mg q12h. All of the adverse events were mild or moderate. There were no deaths or serious or severe adverse events.

### Example 5: Study to Evaluate the Safety and Efficacy of Compound I in Combination Therapy

The safety of Compound I in triple combination with Compounds II and III is evaluated in CF subjects in a 2-part, randomized, double-blind, placebo- and Compound II/III-controlled, parallel-group, multicenter study. Parts 1 and 2 include a Screening Period, a 2-week Treatment Period, and a Safety Follow-up Visit. Part 2 also includes a 4-week Run-in Period before the Treatment Period and a 2-week Washout Period after the Treatment Period.

In previous studies, single doses of Compound I up to 2000 mg and multiple doses of Compound I up to 400 mg q12h (q12h means every twelve hours) were generally safe and well tolerated, except for the occurrence of treatment-emergent hemolysis in a subject who was found to have glucose-6-phosphate dehydrogenase (G6PD) deficiency, and possible occurrence of subclinical hemolysis in a second subject who was also found to have G6PD deficiency. Multiple doses of Compound I up to 300 mg q12h in combination with Compound II (100 mg qd) (qd means once daily) and Compound III (150 mg q12h) were generally safe and tolerated after 14 days of dosing.

### Part 1

In Part 1, three dose levels of Compound I (100, 200, and 300 mg q12h) in triple combination with Compound II (100 mg qd) and Compound III (150 mg q12h) is evaluated in subjects with the F508del/MF genotype.

Part 1 has three cohorts (Cohorts IA, IB, and IC). In Cohort 1A, the triple combination of Compound I at 100 mg q12h, Compound II at 100 mg qd, and Compound III at 150 mg q12h is evaluated in subjects with the F508del/MF genotype. In Cohort IB, the triple combination of Compound I at 200 mg q12h, Compound II at 100 mg qd, and Compound III at 150 mg q12h is evaluated in subjects with the F508del/MF genotype. In Cohort IC, the triple combination of Compound I at 300 mg q12h, Compound II at 100 mg qd, and Compound III at 150 mg q12h is evaluated in subjects with the F508del/MF genotype. Triple placebo is the comparator for all three cohorts

### Part 2

In Part 2, two dose levels of Compound I (200 and 300 mg q12h) in triple combination with Compound II (100 mg qd) and Compound III (150 mg q12h) is evaluated in subjects with the F508del/F508del genotype.

Part 2 has two cohorts (Cohorts 2A and 2B). In Cohort 2A, the triple combination of Compound I at 200 mg q12h, Compound II at 100 mg qd, and Compound III at 150 mg q12h is evaluated in subjects with the F508del/F508del genotype. In Cohort 2B, the triple combination of Compound I at 300 mg q12h, Compound II at 100 mg qd, and Compound III at 150 mg q12h is evaluated in subjects with the F508del/F508del genotype. The combination of placebo, Compound II, and Compound III is the comparator for both cohorts.

TABLE 8
Treatment Arms and Planned Doses for Parts 1 and 2

| | Treatment/ | Compound I | Compound II | Compound III |
|---|---|---|---|---|
| Cohort | Comparator Arms | Dosage | Dosage | Dosage |
| 1A | Treatment | 100 mg qd | 100 mg qd | 150 mg q12h |
| | Comparator | Placebo | Placebo | Placebo |
| IB | Treatment | 200 mg qd | 100 mg qd | 150 mg q12h |
| | Comparator | Placebo | Placebo | Placebo |
| 1C | Treatment | 300 mg qd | 100 mg qd | 150 mg q12h |
| | Comparator | Placebo | Placebo | Placebo |
| 2A^{a} | Treatment | 200 mg qd | 100 mg qd | 150 mg q12h |
| | Comparator | Placebo | 100 mg qd | 150 mg q12h |
| 2B^{a} | Treatment | 300 mg qd | 100 mg qd | 150 mg q12h |
| | Comparator | Placebo | 100 mg qd | 150 mg q12h |

^{a}In Part 2, all subjects will also receive 100 mg qd of Compound II and Compound III 150 mg q12h during (1) a 4 week Run-in Period prior to the 2 week Treatment Period and (2) a 4 week Washout Period following the 2 week Treatment Period.

Primary endpoints for the study include: safety and tolerability assessments based on adverse events (AEs), clinical laboratory values, standard 12-lead electrocardiograms (ECGs), vital signs, and pulse oximetry. Secondary endpoints include: absolute change in sweat chloride concentrations from baseline at Day 15; absolute change in percent predicted forced expiratory volume in 1 second (ppFEV₁) from baseline at Day 15; relative change in ppFEV₁ from baseline at Day 15; absolute change in Cystic Fibrosis Questionnaire-Revised (CFQ-R) respiratory domain score from baseline at Day 15; and PK parameters of Compound I, Compound II, Compound III, and metabolites of Compounds II and III.

### Example 6: Phase 2 Study to Evaluate the Safety and Efficacy Study of Compound I in Combination Therapy

In this Phase 2 randomized, double-blind study, Compound I (100 mg, 200 mg and 300 mg q12h) in combination with Compound II (100 mg qd) and Compound III (150 mg q12h) in people with CF ages 18 and older who have one F508del mutation and one minimal function mutation and in people who have two copies of the F508del mutation was studied. Primary endpoints as described above in Example 6 were for safety and tolerability. Secondary endpoints included absolute change in ppFEV₁ and change in sweat chloride.

Safety Data:

In Part 1 of the study, involving people who had one F508del mutation and one minimal function mutation (F/MF), the triple combination regimen was generally well tolerated. The majority of adverse events were mild or moderate. The most common adverse events (>10%), regardless of treatment group, were infective pulmonary exacerbation of cystic fibrosis, productive cough, diarrhea, cough, headache, sputum increased, and fatigue. There was one drug interruption due to an adverse event in the triple combination treatment group using 200 mg of Compound I and one drug interruption due to an adverse event in the triple combination treatment group using 300 mg of Compound I but none in the control group. An overview of treatment emergent adverse events (TEAEs) is provided in the following table:

| | | Compound I | Compound I | Compound I |
|---|---|---|---|---|
| | | (100 mg q12h) + | (200 mg q12h) + | (300 mg q12h) + |
| | | Compound II | Compound II | Compound II |
| | | (100 mg QD) + | (100 mg QD) + | (100 mg QD) + |
| | | Compound III | Compound III | Compound III |
| | Placebo | (150 mg q12h) | (150 mg q12h) | (150 mg q12h) |
| | (n = 8) | (n = 6) | (n = 10) | (n = 10) |
| Number of TEAEs (Total) | 28 | 13 | 28 | 36 |
| Subjects with any TEAE | 8 (100) | 3 (50) | 7 (70) | 10 (100) |
| Subjects with Related | 1 (13) | 1 (17) | 3 (30) | 6 (60) |
| TEAE* | | | | |
| Subjects with Severe TEAE | 0 | 0 | 0 | 1 (10) |
| Subjects with Serious TEAE | 2 (25) | 0 | 0 | 1 (10) |
| Subjects with TEAE leading | 0 | 0 | 0 | 0 |
| to treatment discontinuation | | | | |
| Subjects with TEAE leading | 0 | 0 | 1 (10) | 1 (10) |
| to drug interruption | | | | |

*Related TEAEs include related and possibly related

Safety Data:

In Part 2 of the study, involving people who had two F508del mutations (F/F), the triple combination regimen was generally well tolerated. No serious or severe adverse events were reported. Two subjects discontinued treatment due to adverse events—one due to neumonia and one due to rash. One subject had a dose interruption due to increased blood bilirubin. An overview of treatment emergent adverse events (TEAEs) is provided in the following table:

| | | Compound I | | Compound I |
|---|---|---|---|---|
| | Placebo + | (200 mg QD) + | Placebo + | (300 mg QD) + |
| | Compound II | Compound II | Compound II | Compound II |
| | (100 mg QD) + | (100 mg QD) + | (100 mg QD) + | (100 mg QD) + |
| | Compound III | Compound III | Compound III | Compound III |
| | (150 mg q12h) | (150 mg q12h) | (150 mg q12h) | (150 mg q12h) |
| | (2 Weeks) | (2 Weeks) | (4 Weeks) | (4 Weeks) |
| | N = 4 | N = 18 | N = 7 | N = 21 |
| Subjects with any TEAE | 3 | 6 | 3 | 19 |
| Subjects with Severe TEAE | 0 | 0 | 0 | 0 |
| Subjects with Serious TEAE | 0 | 0 | 0 | 0 |
| Subjects with TEAE leading to treatment | 0 | 1^{a} | 0 | 1^{b} |
| discontinuation | | | | |
| Subjects with TEAE leading to drug | 0 | 0 | 0 | 1^{c} |
| interruption | | | | |

^{a}Pneumonia
^{b}Rash
^{c}Increased bilirubin

2-Week Efficacy Data in F508del/Minimal Function Patients (F/MF):

In Part 1 of the study, the triple combination was evaluated for two weeks in 34 patients ages 18 and older who had one F508de/mutation and one minimal function mutation (8 in combined placebo, 6 in Compound I 100 mg, 10 in Compound 1 200 mg, and 10 in Compound 1 300 mg). A summary of the within-group ppFEV (primary endpoint) and sweat chloride data (secondary endpoint) through Day 15 is provided below. 2 weeks of treatment with Compound I in triple combination with Compound II and Compound III in subjects who had one F508del mutation and one minimal function mutation resulted in statistically significant (1-sided alpha=5%) and clinically meaningful improvements in ppFEV (5.7-9.7 percentage points), CFQ-R respiratory domain (18.6-21.8 points for 200 and 300 mg of Compound 1 arms), and sweat chloride (13.6-27.5 mmol/L). The treatment was safe and well tolerated with no safety findings of concern.

| Observed Mean Absolute | Observed Mean Absolute at Day | Observed Mean Absolute at Day |
|---|---|---|
| Within-Group Change | 15 Within-Group Change in | 15 Within-Group Change in |
| from Baseline at Day 15* | ppFEV₁ (percentage points) | Sweat Chloride (mmol/L) |
| Triple placebo | −0.8 | −0.1 |
| (n = 8) | (p = 0.6471) | (p = 0.4934) |
| Compound I (100 mg q12h) + | +5.7 | −19.5 |
| Compound II (100 mg QD) + | (p = 0.0095) | (p = 0.0001) |
| Compound III (150 mg q12h) | | |
| (n = 6) | | |
| Compound I (200 mg q12h) + | +9.7 | −13.6^{b} |
| Compound II (100 mg QD) + | (p < 0.0001) | (p = 0.0005) |
| Compound III (150 mg q12h) | | |
| (n = 10) | | |
| Compound I (300 mg q12h) + | +8.0^{a} | −27.5^{b} |
| Compound II (100 mg QD) + | (p = 0.0001) | (p < 0.0001) |
| Compound III (150 mg q12h) | | |
| (n = 10) | | |

*p-values presented are within-group 1-sided p-values
^{a}2 subjects had FEV1 missing at Day 15
^{b}2 subjects had sweat chloride missing at Day 15

2-Week Efficacy Data in F508del Homozygous Patients (F/F):

In Part 2 of the study, the triple combination was evaluated for two weeks in 14 patients ages 18 and older who had two copies of the F508del mutation, who were already receiving the combination of Compound II and Compound III (4 weeks, 4 in placebo and 10 in Compound I 200 mg). A summary of the within-group lung function (ppFEV) (primary endpoint) and sweat chloride data (secondary endpoint) for the triple combination treatment period, from baseline (end of the 4-week Compound II/Compound III run-in period), through Day 15 is provided below.

| Observed Mean Absolute | Observed Mean Absolute at Day | Observed Mean Absolute at Day |
|---|---|---|
| Within-Group Change | 15 Within-Group Change in | 15 Within-Group Change in |
| from Baseline at Day 15* | ppFEV₁ (percentage points) | Sweat Chloride (mmol/L) |
| Placebo + Compound II | −1.0 | +3.5 |
| (100 mg QD) + Compound III | (p = 0.5969) | (p = 0.7176) |
| (150 mg q12h) | | |
| (n = 4) | | |
| Compound I (200 mg q12h) + | +7.3 | −21.3 |
| Compound II (100 mg QD) + | (p = 0.0060) | (p < 0.0001) |
| Compound III (150 mg q12h) | | |
| (n = 10) | | |

*p-values presented are within-group 1-sided p-values

4-Week Efficacy Data in F508del Homozygous Patients (F/F):

A summary of the within-group lung function (ppFEV) (primary endpoint) and sweat chloride data (secondary endpoint) from patients in Part 2 of the study who received the triple combination including 300 mg of Compound I for 4 weeks is provided below.

| Observed Mean Absolute | Observed Mean Absolute at Day | Observed Mean Absolute at Day |
|---|---|---|
| Within-Group Change | 29 Within-Group Change in | 29 Within-Group Change in |
| from Baseline at Day 29* | ppFEV₁ (percentage points) | Sweat Chloride (mmol/L) |
| Placebo + Compound 11 | −2.2 | +1.6 |
| (100 mg QD) + Compound III | (p = 0.8461) | (p = 0.6513) |
| (150 mg q12h) | | |
| (n = 7) | | |
| Compound I (300 mg q12h) + | +6.5 | −22.3 |
| Compound II (100 mg QD) + | (p < 0.0001) | (p < 0.0001) |
| Compound III (150 mg q12h) | | |
| (n = 21) | | |

*p-values presented are within-group 1-sided p-values

In summary, 2-4 weeks of Compound I in triple combination with Compound II and Compound III in homozygous subjects for F508del in Part 2 study resulted in statistically significant and clinically meaningful improvements on top of Compound II and Compound III treatment in ppFEV1 (6.5-7.3 percentage points) and sweat chloride (21.3-22.3 mmol/L). Treatment with Compound I in triple combination with Compound II and Compound III in homozygous subjects for F508del was generally safe and well tolerated; there were no serious AEs and all AEs were mild or moderate.

Summary of 2- and 4-Week Efficacy Data in Parts 1 and 2:

| | Endpoint | Day 15 Results | | | | Day 29 Results | |
|---|---|---|---|---|---|---|---|
| | (Abs. Change | | Compound I | Compound I | Compound I | | Compound I |
| | from Baseline) | Placebo¹ | 100 mg | 200 mg | 300 mg | Placebo¹ | 300 mg |
| Part 1: | n | 8 | 6 | 10 | 10 | — | — |
| F508del/Min. Function | ppFEV1 | −0.8 | 5.7 | 9.7 | 8.0^{a} | — | — |
| (F/MF) | | (p = 0.6471) | (p = 0.0095) | (p < 0.0001) | (p = 0.0001) | | |
| | Sweat | −0.1 | −19.5 | −13.6^{a} | −27.5 | — | — |
| | Chloride | (p = 0.4934) | (p = 0.0001) | (p = 0.0005) | (p < 0.0001) | | |
| Part: 2: | n | 4 | | 10 | 21 | 7 | 21 |
| F508del/F508del | ppFEV1 | −1.0 | | 7.3 | 5.1 | −2.2 | 6.5 |
| (F/F) | | (p = 0.5969) | | (p = 0.0060) | | (p = 0.8461) | (p < 0.0001) |
| | Sweat | | | −21.3 | −23.9 | 1.6 | −22.3 |
| | Chloride | | | (p < 0.0001) | | (p = 0.6513) | (p < 0.0001) |

¹In Part 2, “placebo” was placebo + Compound II (100 mg QD) + Compound III (1.50 mg q12h) as described above.
^{a}Missing date from 2 subjects

### Preclinical Toxicology Data

Preclinical reproductive toxicology studies of Compound I showed no adverse findings of note.

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A method of treating cystic fibrosis comprising administering to a patient in need thereof:
(A) 50 mg to 1000 mg of at least one compound chosen from Compound I 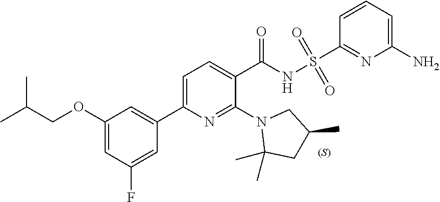
and pharmaceutically acceptable salts thereof daily; and
(B) 25 mg to 200 mg of at least one compound chosen from Compound II: 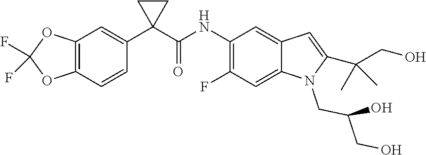 [SMILES: CC(C)(CO)c1cc2cc(NC(=O)C3(c4ccc5c(c4)OC(F)(F)O5)CC3)c(F)cc2n1C[C@@H](O)CO]
and pharmaceutically acceptable salts thereof daily; and
(C) 50 mg to 600 mg of at least one compound chosen from Compound III, Compound III′: 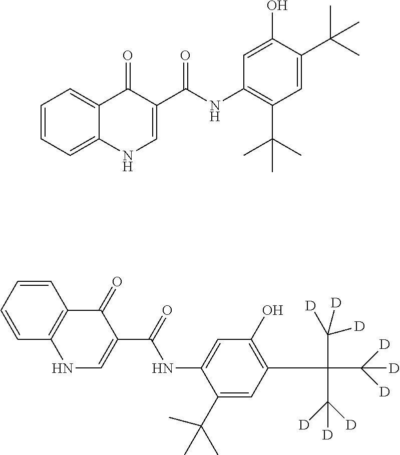 [SMILES: CC(C)(C)c1cc(C(C)(C)C)c(NC(=O)c2c[nH]c3ccccc3c2=O)cc1O ; [2H]C([2H])([2H])C(c1cc(C(C)(C)C)c(NC(=O)c2c[nH]c3ccccc3c2=O)cc1O)(C([2H])([2H])[2H])C([2H])([2H])[2H]]
and pharmaceutically acceptable salts of Compound III or Compound III′ daily.

2. The method according to claim 1, wherein 100 mg of the at least one compound chosen from Compound I and pharmaceutically acceptable salts thereof is administered daily.

3. The method according to claim 1, wherein 200 mg of the at least one compound chosen from Compound I and pharmaceutically acceptable salts thereof is administered daily.

4. The method according to claim 1, wherein 300 mg of the at least one compound chosen from Compound I and pharmaceutically acceptable salts thereof is administered daily.

5. The method according to claim 1, wherein 50 mg to 150 mg of the at least one compound chosen from Compound II and pharmaceutically acceptable salts thereof is administered daily.

6. The method according to claim 5, wherein 50 mg of the at least one compound chosen from Compound II and pharmaceutically acceptable salts thereof is administered daily.

7. The method according to claim 5, wherein 100 mg of the at least one compound chosen from Compound II and pharmaceutically acceptable salts thereof is administered daily.

8. The method according to claim 1, wherein 50 mg to 450 mg of the at least one compound chosen from Compound III, Compound III′, and pharmaceutically acceptable salts of Compound III or Compound III′ is administered daily.

9. The method according to claim 8, wherein 150 mg of the at least one compound chosen from Compound III and pharmaceutically acceptable salts thereof is administered daily.

10. The method according claim 8, wherein 300 mg of the at least one compound chosen from Compound III, Compound III′, and pharmaceutically acceptable salts of Compound III or Compound III′ is administered daily.

11. The method according to claim 1, wherein 100 mg to 300 mg of the at least one compound chosen from Compound I and pharmaceutically acceptable salts thereof is administered daily; 100 mg of the at least one compound chosen from Compound II and pharmaceutically acceptable salts thereof is administered once daily; and 150 mg, 200 mg, or 300 mg of the at least one compound chosen from Compound III, Compound III′, and pharmaceutically acceptable salts of Compound III or Compound III′ is administered daily.

12. The method according to claim 1, wherein 100 mg to 300 mg of the at least one compound chosen from Compound I and pharmaceutically acceptable salts thereof is administered daily; 50 mg per dose of the at least one compound chosen from Compound II and pharmaceutically acceptable salts thereof is administered twice daily; and 150 mg per dose of the at least one compound chosen from Compound III and pharmaceutically acceptable salts thereof is administered twice daily.

13. The method according to claim 1, wherein 100 mg, 200 mg, or 300 mg per dose of the at least one compound chosen from Compound I and pharmaceutically acceptable salts thereof is administered twice daily; 100 mg of Compound II is administered once daily; and 150 mg per dose of Compound III is administered once or twice daily.

14. The method according to claim 1, wherein 100 mg, 200 mg, or 300 mg per dose of the at least one compound chosen from Compound I and pharmaceutically acceptable salts thereof is administered twice daily; 50 mg per dose of Compound II is administered twice daily; and 75 mg per dose of Compound III is administered twice daily.

15. The method according to claim 1, wherein 100 mg, 200 mg, or 300 mg of the at least one compound chosen from Compound I and pharmaceutically acceptable salts thereof is administered daily; 100 mg of Compound II is administered daily; and 200 mg of Compound III′ is administered daily.

16. (canceled)

17. (canceled)

18. (canceled)

19. (canceled)

20. (canceled)

21. A pharmaceutical composition comprising:
(A) 100 mg, 200 mg, or 300 mg of at least one compound chosen from Compound I and pharmaceutically acceptable salts thereof: 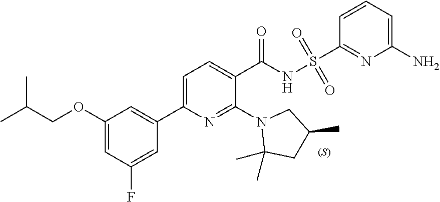
(B) 100 mg or 50 mg of at least one compound chosen from Compound II and pharmaceutically acceptable salts thereof: 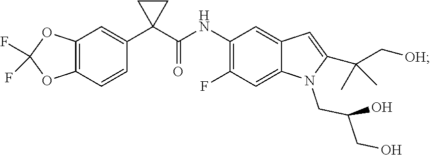 [SMILES: CC(C)(CO)c1cc2cc(NC(=O)C3(c4ccc5c(c4)OC(F)(F)O5)CC3)c(F)cc2n1C[C@@H](O)CO]
(C) 75 mg, 150 mg, 200 mg, or 300 mg of at least one compound chosen from Compound III, Compound III′, and pharmaceutically acceptable salts of Compound III or Compound III′:

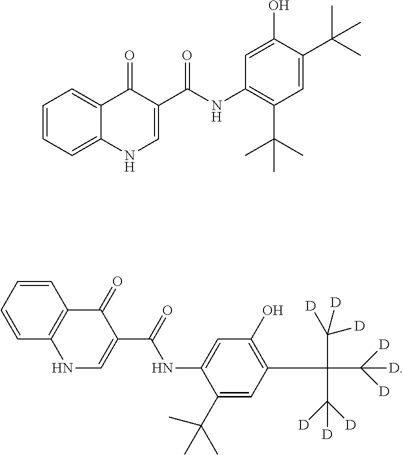

22. A pharmaceutical composition according to claim 21 comprising:
(A) 100 mg of at least one compound chosen from Compound I and pharmaceutically acceptable salts thereof;
(B) 50 mg of at least one compound chosen from Compound II and pharmaceutically acceptable salts thereof; and
(C) 75 mg of at least one compound chosen from Compound III, Compound III′, and pharmaceutically acceptable salts of Compound III or Compound III′.

23. A pharmaceutical composition according to claim 21 comprising:
(A) 200 mg of at least one compound chosen from Compound I and pharmaceutically acceptable salts thereof;
(B) 100 mg of at least one compound chosen from Compound II and pharmaceutically acceptable salts thereof; and
(C) 150 mg of at least one compound chosen from Compound III, Compound III′, and pharmaceutically acceptable salts of Compound III or Compound III′.

24. A pharmaceutical composition according to claim 21 comprising:
(A) 200 mg of at least one compound chosen from Compound I and pharmaceutically acceptable salts thereof;
(B) 100 mg of at least one compound chosen from Compound II and pharmaceutically acceptable salts thereof; and
(C) 150 mg of at least one compound chosen from Compound III and pharmaceutically acceptable salts thereof.

25. A pharmaceutical composition according to claim 21 comprising:
(A) 200 mg of at least one compound chosen from Compound I and pharmaceutically acceptable salts thereof;
(B) 100 mg of at least one compound chosen from Compound II and pharmaceutically acceptable salts thereof; and
(C) 150 mg or 200 mg of at least one compound chosen from Compound III′ and pharmaceutically acceptable salts thereof.

26. A pharmaceutical composition according to claim 21 comprising:
(A) 300 mg of at least one compound chosen from Compound I and pharmaceutically acceptable salts thereof;
(B) 200 mg of at least one compound chosen from Compound II and pharmaceutically acceptable salts thereof; and
(C) 150 mg or 200 mg of at least one compound chosen from Compound III, Compound III′, and pharmaceutically acceptable salts of Compound III or Compound III′.
